(19) United States
(12) Patent Application Publication
(10) Pub. No.: US 2010/0178305 A1
(43) Pub. Date: Jul. 15, 2010

(54) ULTRASONIC NANOTHERAPY OF SOLID TUMORS WITH BLOCK COPOLYMERS STABILIZED PERFLUOROCARBON NANODROPLETS

(75) Inventor: Natalya (Natalia) RAPOPORT, Sandy, UT (US)

Correspondence Address: GARDNER GROFF GREENWALD & VILLANUEVA. PC, 2018 POWERS FERRY ROAD, SUITE 800, ATLANTA, GA, 30339, US

(73) Assignee: UNIVERSITY OF UTAH RESEARCH FOUNDATION, Salt Lake City, UT (US)

(21) Appl. No.: 12/632,254

(22) Filed: Dec. 7, 2009

Related U.S. Application Data
(60) Provisional application No. 61/122,558, filed on Dec. 15, 2008.

(51) Int. Cl. A61K 31/337; A61P 35/00; A61K 9/10; A61K 31/351; A61K 31/7068; A61K 33/24; A61K 31/5513; A61K 31/7048; A61K 31/56

(52) U.S. Cl. 424/400; 514/449; 514/459; 514/49; 424/649; 514/221; 514/31; 514/178; 977/788

(57) ABSTRACT

Described herein are methods of treating a tumor by contacting the tumor with a therapeutic agent encapsulated in a first nanoemulsion and exposing the tumor to a first ultrasonic radiation of less than 300 kHz to the tumor. In some aspects, the tumor can be contacted with a second nanoemulsion. In some aspects, the second emulsion can be injected directly into the tumor via intratumoral injection before exposing the tumor to the first ultrasonic radiation. In some aspects, the tumor is exposed to a second ultrasonic radiation from about 1 to 5 MHz after the first ultrasonic radiation. The methods described herein can be used to treat numerous tumors including, but not limited to, multidrug resistant tumors and inoperable tumors. These tumors may include, but are not limited to, breast cancers, ovarian cancers, pancreatic cancers, or any combination thereof.

### CROSS-REFERENCE TO RELATED APPLICATIONS

This application claims benefit of U.S. Provisional Application Ser. No. 61/122,558 filed on Dec. 15, 2008, which is hereby incorporated by reference in its entirety for all of its teachings.

### ACKNOWLEDGEMENT

The research leading to this invention was funded in part by the National Institutes of Health, Grant No. R01EB 1033. The Government has certain rights in this invention.

### BACKGROUND

Severe side effects of current tumor chemotherapy are caused by drug attack on healthy tissues. To solve systemic toxicity problems, various drug delivery modalities have been suggested that are commonly based on drug encapsulation in carriers such as liposomes, polymeric micelles, and hollow nanocontainers. These drug carriers are targeted to tumors either passively or actively. Tumor targeting of many carriers is often inefficient because the carriers are too large to extravasate through the inter-endothelial gaps of the tumor and drug release of the drugs contained within the carriers is often problematic. Disclosed herein are methods of using therapeutic agents encapsulated in a nanoemulsion coupled with the use of ultrasonic radiation to treat tumors.

### SUMMARY OF EMBODIMENTS

Described herein are methods of treating a tumor by contacting the tumor with a therapeutic agent encapsulated in a first nanoemulsion and exposing the tumor to a first ultrasonic radiation of less than 300 kHz to the tumor. In some aspects, the tumor can be contacted with a second nanoemulsion. In some aspects, the second nanoemulsion can be injected directly into the tumor via intratumoral injection before exposing the tumor to the first ultrasonic radiation. In some aspects, the tumor is exposed to a second ultrasonic radiation from about 1 to 5 MHz after the first ultrasonic radiation. The methods described herein can be used to treat numerous tumors including, but not limited to, multidrug resistant tumors and inoperable tumors. These tumors may include, but are not limited to, breast cancers, ovarian cancers, pancreatic cancers, or any combination thereof. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows a schematic representation of a passive drug targeting through the defective tumor microvasculature. The formulation comprises polymeric micelles (small circles), nanodroplets (stars), and microbubbles (large circles); micelles are formed by a biodegradable block copolymer (e.g. PEG-PLLA or PEG-PCL or Pluronic or mixtures thereof); bubbles are formed by perfluorocarbon (e.g. PFP) stabilized by the same (or different) biodegradable block copolymer. Lipophilic drug (e.g. DOX or PAX) is localized in the micelle cores or in the walls of nano/microbubbles.

FIG. 2A shows the particle size distribution in (a) 0.25% PEG-PLLA; (b) Genexol-PM/0.25% PEG-PLLA; (c) 1% PFP/0.25% PEG-PLLA; and (d) Genexol-PM/1% PFP/0.25% PEG-PLLA.

FIG. 3 shows the top view of the experimental set used for measurements of the acoustic properties of nanodroplets/microbubbles described herein.

FIG. 4 shows photographs of a mouse bearing two ovarian carcinoma tumors before (left) and three weeks after the treatment (right); a mouse was treated by four systemic injections of nanodroplet-encapsulated paclitaxel nbGEN (20 mg/kg as paclitaxel) given twice weekly; the right tumor was sonicated by 1-MHz CW ultrasound (peak-to-peak pressure 1.18 MPa, exposure duration 1 min) delivered 4.5 hours after the injection of the drug formulation through a water bag coupled to a transducer and mouse skin by Aquasonic coupling gel.

FIG. 5 shows the ADV effect in a 1% PFP/0.25% PEG-PLLA nanoemulsion inserted in a plasma clot; (A)—initial gel; (B)—gel sonicated by 1-MHz CW ultrasound for 1 min at 1.18 MPa; (C)—gel sonicated by 90-kHz ultrasound for 1 min at 0.7 MPa.

FIG. 6 shows stable cavitations as characterized by relative second harmonic (A, B) or subharmonic (C,D) amplitudes generated by the microbubbles in PBS (A, C) or agarose gel (B, D); the frequency is 1 MHz.

FIG. 7 (A-F) shows stable and inertial cavitation thresholds observed in PBS and gel systems in experiments with focused ultrasound beam generated by a HIFU transducer; ultrasound beam was focused in the sample volume that mostly avoided the pre-existing bubbles. Specifically, FIGS. 7 (A-D) show stable cavitation as characterized by relative second harmonic (A, B) or subharmonic (C,D) amplitudes, and FIGS. 7 (E-F) show inertial cavitation as characterized by broadband noise (E,F) amplitudes generated by the microbubbles in PBS (A, C, E) or agarose gel (B, D, F) under HIFU ultrasound; the frequency is 1 MHz.

FIG. 8 shows (A)—Fluorescence images of the 0.75 mg/ml Dox/0.5% PEG-PLLA/2% PFP formulation placed in a plastic capillary (internal diameter 340 um) of a snake mixer slide (XXS, Zweibrucken, Germany). (B) Fluorescence image of the MDA MB231 cell (thick arrow) and bubble (thin arrow) aggregates formed in the capillary during a 150-s sonication (corresponding to a 30-s ultrasound exposure time) by 3-MHz ultrasound at a 2 W/cm² nominal power density with a 20% duty cycle, with the focus on the bubbles. Ultrasound was applied directly to the slide through the Aquasonic coupling gel. (C) Fluorescence image of another cell aggregate of the same sample, with focus on the cells.

FIG. 9 (A-C) shows effective regression of an ovarian carcinoma tumor in a nude mouse treated by systemic injections of 1% PFP/0.25% PEG-PLLA nanoemulsion formulation of PTX, nbGEN (20 mg/kg as PTX) combined with ultrasound; two treatment rounds were given with a two-week break between the rounds; within each treatment round, drug injection/sonication was given twice weekly for two weeks; unfocused CW1-MHz ultrasound was applied locally to the tumor for 60 s four hours after the drug injection at a peak-to-peak pressure of 1.18 MPa. The first photograph (FIG. 9(A)) was taken before the start of the treatment, the second (FIG. 9(B))—two weeks later, i.e. immediately after the last treatment of the first treatment round. The third photograph (FIG. 9(C)) was taken one week after completion of the first treatment round. FIG. 9(D) shows normalized tumor growth/regression curve for the mouse presented in FIG. 9(A).

FIG. 10 shows (A)—Four B-mode ultrasound images of nanodroplets and bubbles in Agarose gel; bubbles were formed after injection of 1% PFP/0.25% PEG-PCL nanoemulsion through a 26-gage needle; images were taken consecutively during the first 90 s after injection. (B)—B-mode ultrasound image of a pancreatic tumor after direct injection of nbGEN. Images were taken with a 14-MHz linear transducer Acuson Sequoia.

FIG. 11 shows B-mode ultrasound images of the orthotopic pancreatic tumor (A)—before and (B)—5 h after systemic injection of paclitaxel-loaded nanodroplets (nbGEN).

FIG. 12 shows B-mode ultrasound images of the same slice of a subcutaneous pancreatic tumor (A)—before and (B)—after tumor sonication for 30 s by 90-kHz ultrasound at a pressure of 0.7 MPa; sonication was performed and images were taken 4.5 h after the injection of nbGEN; after sonication, the brightness of some specks increased by about 30% and new bright specks appeared in the slice (indicated by arrows) manifesting ultrasound-induced formation of larger bubbles in tumor tissue. The brightness of the lower spot indicated by arrow went up from 112 to 147 relative units; the brightness of the upper spot indicated by arrow went up from 92 to 118 relative units; (K—kidney).

FIG. 13 shows pancreatic tumor growth curves for animals treated by Gemcitabine (GEM), micellar formulation of paclitaxel Genexol PM (GEN), combination drug Genexol PM+GEM, and nanoemulsion formulation of paclitaxel nbGEN+GEM and ultrasound.

FIG. 14 shows a growth/regression curve of a large pancreatic tumor treated with combination drug GEM+nbGEN combined with tumor sonication by continuous wave 1-MHz ultrasound applied for 30 s at 3.4 W/cm² nominal power density to the mouse abdominal area in the pancreas region. Arrows indicate days of treatment.

FIG. 15 shows suppression of metastasis by the ultrasound-mediated chemotherapy of pancreatic cancer using micellar or nanodroplet encapsulated paclitaxel (N=6).

FIG. 16 shows an ultrasound image of the control pancreatic tumor. MASS—tumor; ASC—ascites; SPL—spleen.

FIG. 17 shows tumor growth curves for mice treated with GEM (open symbols) or nbGEN+GEM+US (closed symbols). Different symbols correspond to different animals. Intra-group variation is larger for a nanodroplet encapsulated drug than for a molecularly dissolved drug.

FIG. 18 shows a grayscale distribution in four slices of the same tumor recorded 5 h after the systemic injection of the nanodroplet encapsulated paclitaxel (nbGEN); the images manifest non-uniform nanodroplet distribution in tumor tissue.

FIG. 19 shows a power doppler image of the subcutaneous control pancreatic tumor (A) and Color Doppler image of the orthotopic pancreatic tumor recorded 5 h after the systemic injection of the nanodroplet encapsulated paclitaxel (B). Vascularization is visible at the periphery or around the tumor. The increase in tumor echogenicity manifests the nanodroplet accumulation.

FIG. 20 shows growth of Gemcitabin (GEM)-resistant MiaPaCa2 pancreatic cancer cells in GEM-containing medium in hyperthermia conditions (43° C.) in the absence (A) or presence (B) of drug resistance suppressor Pluronic L-61 (0.1%) incorporated in 0.25% PEG-PLLA micelles.

FIG. 21(a) shows a mouse model mimicking large inoperable tumors and treating the tumors with the methods described herein. FIG. 21(b) shows an ultrasound image of a MDA MB231 breast cancer tumor grown subcutaneously in a nu/nu mouse; the image was taken a month after direct intratumoral injection of a 100 μl of a 1% PEP/0.25% PEG-PCL nanoemulsion. The image shows that the microbubbles were preserved in tumor tissue for at least a month. This suggests that one direct intratumoral injection of a nanoemulsion may allow for multiple uses as a catalyst of ultrasound enhanced droplet-to-bubble transition of systemically injected nanodroplets after the systemically injected nanodroplets have accumulated in the tumor tissue.

FIG. 22 shows a time lapse photo of PFP/PEG-PLLA nanodroplets in a plasma clot either before exposure to ultrasonic radiation or 1 minute after, 2 minutes after, or 6 minutes after being exposed to 20 kHz ultrasonic radiation for 30 seconds.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a therapeutic agent” includes mixtures of two or more such therapeutic agents, and the like.

“Optional” or “optionally” means that the subsequently described event or circumstance can or cannot occur, and that the description includes instances where the event or circumstance occurs and instances where it does not. For example, the phrase “optionally a second therapeutic agent” means that the second therapeutic agent may or may not be present in the compositions used for the methods described herein.

“Treating” or “treatment” refers to the reduction of tumor growth, the prevention of tumor growth, the eradication (i.e., killing) or a tumor, and/or the reduction in size of tumors and cancers as described herein.

“Therapeutic agent” refers to a chemical compound, a hormone, or a biological molecule including nucleic acids, peptides, proteins, and antibodies that can be used to reduce or prevent a condition.

“Nanoemulsion” refers to a system that includes micelles and/or nanodroplets in which the micelles and nanodroplets are less than 1500 nm, or more preferably less than 1000 nm in diameter, which are capable of encapsulating a therapeutic agent.

“Subject” refers to mammals including, but not limited to, humans, non-human primates, sheep, dogs, rodents (e.g., mouse, rat, etc.), guinea pigs, cats, rabbits, cows, and non-mammals including chickens, amphibians, and reptiles, who are at risk for or have been diagnosed with a tumor and benefits from the methods and compositions described herein.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “alkylene group” as used herein is a branched or unbranched unsaturated hydrocarbon group of 1 to 24 carbon atoms such as methylene, ethylene, propene, butylene, isobutylene and the like.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, halo, hydroxy, alkylthio, arylthio, alkoxy, aryloxy, amino, mono- or di-substituted amino, ammonio or substituted ammonio, nitroso, cyano, sulfonato, mercapto, nitro, oxo, alkyl, alkenyl, cycloalkyl, benzyl, phenyl, substituted benzyl, substituted phenyl, benzylcarbonyl, phenylcarbonyl, saccharides, substituted benzylcarbonyl, substituted phenylcarbonyl and phosphorus derivatives. The aryl group can include two or more fused rings, where at least one of the rings is an aromatic ring. Examples include naphthalene, anthracene, and other fused aromatic compounds.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

It is understood that any given particular aspect of the disclosed compositions and methods can be easily compared to the specific examples and embodiments disclosed herein. By performing such a comparison, the relative efficacy of each particular embodiment can be easily determined Particularly preferred compositions and methods are disclosed in the Examples herein, and it is understood that these compositions and methods, while not necessarily limiting, can be performed with any of the compositions and methods disclosed herein.

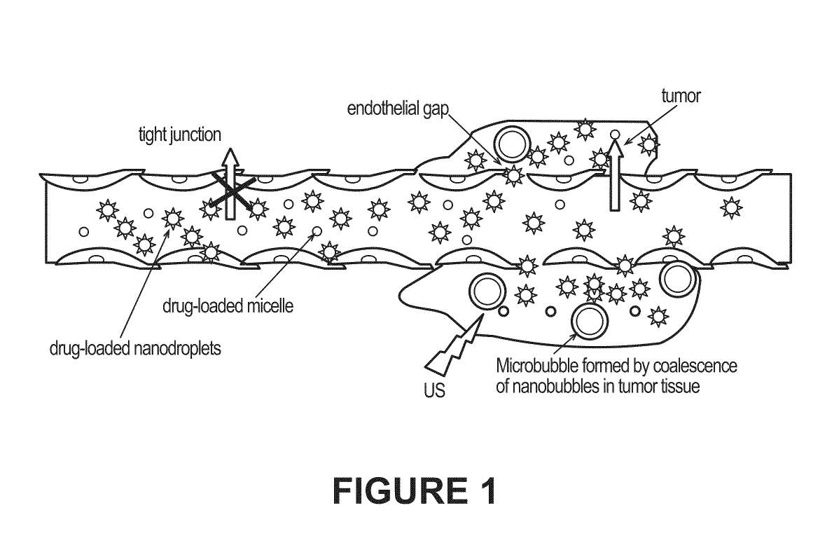

Described herein are nanoemulsions containing one or more therapeutic agents that accumulate and/or extravasate in tumors via, for example, the enhance permeability and retention (EPR) effect. As shown in FIG. 1, these nanoemulsions can be subsequently converted into microbubbles in situ with ultrasound-triggered drug release. The conversion from a nanoemulsion, which includes nanodroplets, to microbubbles is known as acoustic droplet vaporization (ADV). The compositions and methods described herein may present an efficient double-targeting chemotherapeutic modality for solid tumors. In some aspects, the methods of treating tumors as described herein can be performed by contacting the tumor with a therapeutic agent encapsulated in a first nanoemulsion and exposing the tumor to a first ultrasonic radiation at a particular frequency. In some aspects, the tumor can include a multidrug resistant tumor, an inoperable tumor, or a combination thereof. In certain aspects, the tumor includes, but is not limited to breast cancer, pancreatic cancer, ovarian cancer, or a combination thereof. In one aspect, the tumor is pancreatic ductal adenocarcinoma (PDA). In this aspect, most PDA presentations are inoperable at the time of diagnosis due to the extensive tumor burden, local invasion, poor general health, and multiple aggressive micrometastases that are resistant to chemotherapy and radiation treatment. About 40% of patients have a dismal prognosis; median survival time is only 3-6 months. The only FDA-approved treatment for PDA is the administration of the nucleoside analogue gemcitabine (GEM), but the partial response rate to chemotherapy is well below 10%, which is most likely due to the development of GEM resistance during the course of chemotherapy.

Therefore, subjects diagnosed with PDA can benefit from the methods described herein due to more efficient delivery of a therapeutic agent to the PDA.

In some aspects, the tumor as described above can be contacted with a first nanoemulsion by direct injection into the tumor, by subcutaneous injection, by intramuscular injection, or via systemic injection of the nanoemulsion, which includes intravenous injection. When the nanoemulsion is administered systemically, adequate time is given for the nanoemulsion to extravasate into the tumor before exposing the tumor to the first ultrasonic radiation. In some aspects, a time ranging from about 4 hours to about 24 hours is provided to allow the nanoemulsion to extravasate into the tumor. In yet another aspect, a time ranging from about 4 hours to about 8 hours, from about 8 hours to about 14 hours or from about 10 hours to about 24 hours is provided to allow the nanoemulsion to extravasate into the tumor.

In certain aspects, a second nanoemulsion can contacted with the tumor. For example, the second nanoemulsions can be directly injected into the tumor via intratumoral injection before exposing the tumor to a first ultrasonic radiation. In some aspects, a second nanoemulsion can be directly injected into the tumor after contacting the tumor with the first nanoemulsion but before exposing the tumor to a first ultrasonic radiation. In some aspects, the tumor is immediately exposed to the first ultrasonic radiation after being directly injected with the second nanoemulsion.

For example, the methods described herein include treating a tumor by the following steps: (a) contacting the tumor with a therapeutic agent encapsulated in a first nanoemulsion; and (b) exposing the tumor to a first ultrasonic radiation in an amount less than 100 kHz. In some aspects, a second nanoemulsion can be directly injected into the tumor (i.e., via intratumoral injection) after step (a) but before step (b). In some aspects, the second nanoemulsion can be directly injected into the tumor before step (b).

After the tumor has been contacted with the first nanoemulsion and/or second nanoemulsion as described above (e.g., nanoemulsions administered to a subject), the tumor is exposed to a first ultrasonic radiation having a relatively low frequency. For example, this low frequency ultrasonic radiation can aid in conversion of the nanodroplet(s) to microbubble(s) (i.e., ADV), which can allow for more efficient delivery of the therapeutic agent to the tumor. In some aspects, this first ultrasonic radiation can be high intensity focused ultrasound (HIFU) radiation, continuous wave (CW) ultrasound radiation, pulsed waved (PW) ultrasound radiation, or any combination thereof. In some aspects, the ultrasonic radiation can be applied by using low-frequency therapeutic ultrasound transducers. In some aspects, the first ultrasonic radiation frequency can be less than or equal to 400 kHz, 375 kHz, 350 kHz, 325 kHz, 300 kHz, 275 kHz, 250 kHz, 225 kHz, 200 kHz, 175 kHz, 150 kHz, 125 kHz, 100 kHz, 95 kHz, 90 kHz, 85 kHz, 80 kHz, 75 kHz, 70 kHz, 65 kHz, 60 kHz, 55 kHz, 50 kHz, 45 kHz, 40 kHz, 35 kHz, 30 kHz, 25 kHz, or 20 kHz. In some aspects, the first ultrasonic radiation frequency is less than 100 kHz. In some aspects, the first ultrasonic radiation frequency is less than 95 kHz. In one aspect, the first ultrasonic radiation frequency is less than or equal to 90 kHz. In some aspects, the first ultrasonic radiation is from about 20 kHz to about 90 kHz, wherein the frequency can vary by about plus or minus 5 kHz. In one aspect the first ultrasonic radiation is about 90 kHz. In some aspects, the first ultrasonic radiation frequency has a peak to peak pressure ranging from about 0.5 MPa to about 2 MPa. In some aspects, the first ultrasonic radiation frequency has a peak to peak pressure ranging from about 0.7 MPa to about 1.5 MPa.

Following the first ultrasonic radiation step, in some aspects, the tumor can be exposed to a second ultrasonic radiation. For example, this second ultrasonic radiation can be high intensity focused ultrasound (HIFU) radiation, continuous wave (CW) ultrasound radiation, pulsed waved (PW) ultrasound radiation, or any combination thereof that has varying frequencies. In this aspect, the second ultrasonic radiation can be less than or equal to about 15 MHz, 14.5 MHz, 14 MHz, 13.5 MHz, 13 MHz, 12.5 MHz, 12 MHz, 11.5 MHz, 11 MHz, 10.5 MHz, 10 MHz, 9.5 MHz, 9.0 MHz, 8.5 MHz, 8.0 MHz, 7.5 MHz, 7.0 MHz, 6.5 MHz, 6.0 MHz, 5.5 MHz, 5.0 MHz, 4.5 MHz, 4.0 MHz, 3.5 MHz, 3.0 MHz, 2.5 MHz, 2.0 MHz, 1.5 MHz, 1.0 MHz, or 0.5 MHz. In some aspects, the second ultrasonic radiation frequency ranges from about 1 MHz to about 5 MHz. In some aspects, the second ultrasonic radiation frequency ranges from about 1 MHz to about 4 MHz, from about 1 MHz to about 3 MHz, or from about 1 MHz to about 2 MHz. In one aspect, the second ultrasonic radiation frequency is less than about 1.5 MHz or is greater than or equal to 1 MHz. In some aspects, the second ultrasonic radiation frequency has a peak to peak pressure ranging from about 0.5 MPa to about 7 MPa. In some aspects, the second ultrasonic radiation frequency has a peak to peak pressure ranging from about 1 MPa to about 3 MPa.

As stated above, at least one therapeutic agent can be encapsulated within the first nanoemulsion and if desired within the second nanoemulsion. In this aspect, the therapeutic agent can include lipophilic drugs that have a low aqueous solubility. For example, these therapeutic agents can include chemotherapeutic drugs, hormones, or any other biologically or chemically active drugs, which include nucleic acids, peptides, proteins, and/or antibodies, that can be used to treat a condition such as various tumors and cancers. In some aspects, the therapeutic agent can include, but is not limited to, paclitaxel, doxorubicin, gemcitabine, adriamycin, cisplatin, taxol, methotrexate, 5-fluorouracil, betulinic acid, amphotericin B, diazepam, nystatin, propofol, testosterone, estrogen, prednisolone, prednisone, 2,3 mercaptopropanol, progesterone, multi-drug resistant (MDR) suppressing agents, or any combination thereof. In some aspects, MDR suppressing agents include, but are not limited to, verapamil, the Cyclosporin A analogue PCS 833, oligodeoxynucleotides (ODNs), ribozimes, valspodar (PSC833), curcumin (CUR), pluronic L-61, pluronic 105, pluronic 85 or any combination thereof. In some aspects, the therapeutic agent can include, but is not limited to, paclitaxel, doxorubicin, gemcitabine, or any combination thereof. For example, in one aspect the therapeutic agent encapsulated in the nanoemulsion can include only paclitaxel. In some aspects, the therapeutic agent encapsulated in the nanoemulsion includes at least paclitaxel. In another aspect, the therapeutic agent encapsulated in the nanoemulsion can include only doxorubicin. In some aspects, the therapeutic agent encapsulated in the nanoemulsion includes at least doxorubicin. In yet another aspect, the therapeutic agent encapsulated in the nanoemulsion can include only gemcitabine. In some aspects, the therapeutic agent encapsulated in the nanoemulsion includes at least gemcitabine. In some aspects, the therapeutic agent encapsulated in the nanoemulsion includes at least paclitaxel and doxorubicin. In some aspects, the therapeutic agent encapsulated in the nanoemulsion can include at least paclitaxel and gemcitabine. In some aspects, the therapeutic agent encapsulated in the nanoemulsion can include at least doxorubicin and gemcitabine. In yet another aspect, the therapeutic agent encapsulated in the nanoemulsion includes at least paclitaxel, doxorubicin, and gemcitabine.

The nanoemulsions described herein (i.e., the first and second nanoemulsions) can be amphiphilic compositions that have a hydrophilic outer surface and a lipophilic inner core. These nanoemulsions make it possible to efficiently transport lipophilic therapeutic agents or drugs to tumors, and due to the tumor's vasculature, these nanoemulsions, which include therapeutic agents encapsulated within the nanoemulsions, can be easily extravasated into the tumor. In some aspects, the nanoemulsions include nanosized micelles and nanodroplets that have diameters that are less than about 1500 nm, about 1400 nm, about 1300 nm, about 1200 nm, about 1100 nm, about 1050 nm, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 250 nm, about 200 nm, about 150 nm, about 100 nm, about 90 nm, about 80 nm, about 70 nm, about 60 nm, about 50 nm, about 40 nm, about 30 nm, about 20 nm, or about 10 nm. In some aspects, the micelles have diameters ranging from about 20 nm to about 150 nm. In some aspects, the micelles have diameters ranging from about 20 nm to about 100 nm. In some aspects, the nanodroplets have diameters ranging from about 90 nm to about 1200 nm. In some aspects, the nanodroplets have diameters ranging from about 100 nm to about 800 nm.

As described above, the nanoemulsions can be amphiphilic compositions. In some aspects, the nanoemulsions (i.e., the first and/or the second nanoemulsions) can independently include, but are not limited to, a block copolymer, a halogen containing compound, or a combination thereof. For example, in certain aspects, the nanoemulsion can include a block copolymer and at least one therapeutic agent encapsulated within the nanoemulsion. In yet another example, the nanoemulsion can include a block copolymer, a halogen containing compound, and at least one therapeutic agent encapsulated within the nanoemulsion.

In some aspects, the block copolymer described herein can include, but is not limited to, a hydrophilic polymer (i.e., a hydrophilic block) and a second polymer. In some aspects, the hydrophilic polymer can include a poly(alkylene oxide), a polyvinyl polymer such as polyvinyl pyrrolidone, or any combination thereof. In some aspects, the hydrophilic polymer, which includes poly(alkylene oxides), can have a molecular weight ranging from 0 to 1000 Da, from 0 to 1500 Da, from 0 to 2000 Da, from 0 to 2500 Da, from 0 to 3000 Da, from 0 to 3500 Da, from 0 to 4000 Da, from 0 to 4500 Da, or from 0 to 5000 Da. In some aspects, the poly(alkylene oxide) can include a polyethylene oxide, a polypropylene oxide, a polybutylene oxide, a polypentylene oxide, or a combination thereof. In one aspect, the poly(alkylene oxide) is polyethylene oxide. In this aspect, the polyethylene glycol can include a molecular weight ranging from 0 to 1000 Da, from 0 to 2000 Da, from 0 to 3000 Da, from 0 to 4000 Da, or from 0 to 5000 Da.

In some aspects, the second polymer of the block copolymer is a hydrophobic polymer (i.e., a hydrophobic block). In this aspect, the hydrophobic polymer should preferably be biodegradable and biocompatible. In some aspects, the hydrophobic polymer can have a molecular weight ranging from 0 to 1000 Da, from 0 to 1500 Da, from 0 to 2000 Da, from 0 to 2500 Da, from 0 to 3000 Da, from 0 to 3500 Da, from 0 to 4000 Da, from 0 to 4500 Da, from 0 to 5000 Da, from 0 to 5500 Da, from 0 to 6000 Da, from 0 to 6500 Da, from 0 to 7000 Da, from 0 to 7500 Da, from 0 to 8000 Da, from 0 to 8500 Da, from 0 to 9000 Da, from 0 to 9500 Da, from 0 to 10000 Da, from 0 to 10500 Da, from 0 to 11000 Da, from 0 to 115000 Da, or from 0 to 12000 Da. In this aspect, the second polymer can include, but is not limited to, a polymer of lactic acid, a polylactone, or a combination thereof. Examples of the lactic acid that are present in the block copolymer can include a poly(l)lactic acid, a poly(d,l)lactic acid, or a combination thereof. In some aspects, the molecular weight of the poly(l)lactic acid, poly(d,l)lactic acid, or a combination thereof can range from 0 to 1000 Da, from 0 to 1500 Da, from 0 to 2000 Da, from 0 to 2500 Da, from 0 to 3000 Da, from 0 to 3500 Da, from 0 to 4000 Da, from 0 to 4500 Da, from 0 to 5000 Da, from 0 to 5500 Da, from 0 to 6000 Da, from 0 to 6500 Da, from 0 to 7000 Da, from 0 to 7500 Da, from 0 to 8000 Da, from 0 to 8500 Da, from 0 to 9000 Da, from 0 to 9500 Da, from 0 to 10000 Da, from 0 to 10500 Da, from 0 to 11000 Da, from 0 to 115000 Da, or from 0 to 12000 Da. In some aspects, the molecular weight of the poly(l)lactic acid, poly(d,l)lactic acid, or a combination thereof can range from 4000 Da to 5000 Da in molecular weight. In one aspect, the second polymer is poly(l)lactic acid having a molecular weight of 4700 Da. Examples of the lactone that are present in the block copolymer can include polycaprolactone. In some aspects, the molecular weight of the polycaprolactone can range from 0 to 1000 Da, from 0 to 1500 Da, from 0 to 2000 Da, from 0 to 2500 Da, from 0 to 3000 Da, from 0 to 3500 Da, from 0 to 4000 Da, from 0 to 4500 Da, from 0 to 5000 Da, from 0 to 5500 Da, from 0 to 6000 Da, from 0 to 6500 Da, from 0 to 7000 Da, from 0 to 7500 Da, from 0 to 8000 Da, from 0 to 8500 Da, from 0 to 9000 Da, from 0 to 9500 Da, from 0 to 10000 Da, from 0 to 10500 Da, from 0 to 11000 Da, from 0 to 115000 Da, or from 0 to 12000 Da. In some aspects, the molecular weight of the polycaprolactone can range from 2000 Da to 3000 Da. In one aspect, the second polymer is polycaprolactone having a molecular weight of 2600 Da.

The nanoemulsions described herein can also contain a halogen containing compound. For example, the halogen containing compound has at least one halogen group wherein the at least one halogen group can include at least one of the following: at least one fluorine group, at least one chlorine group, at least one bromine group, at least one iodine group, at least one astatine group, at least one ununseptium group, or any combination thereof. In some aspects, the halogen containing compound can be a halogenated alkane, a halogenated cycloalkane, a halogenated alkylene, a halogenated cycloalkylene, a halogenated alkyne, a halogenated aryl, a halogenated heterocycle, or any combination thereof. In one aspect, the halogen containing compound includes a fluoro containing compound. For example, the fluoro containing compound can include perfluorocarbons such as a perfluoroalkyl, a perfluorocycloalkyl, a perfluoroalkylene, or a perfluoroalkynes. Examples of perfluoroalkyl compounds and examples of perfluorocycloalkyl compounds include, but are not limited to, perfluoromethane, perfluoroethane, perfluoropropane, perfluorocyclopropane, perfluorobutane, perfluorocyclobutane, perfluoropentane, perfluorocyclopentane, perfluorohexane, perfluorohexane, perfluoroheptane, perfluorocycloheptane, perfluorooctane, perfluorocyclooctane, perfluorononane, and perfluorodecane. In some aspects, the halogen containing compound is perfluoropentane.

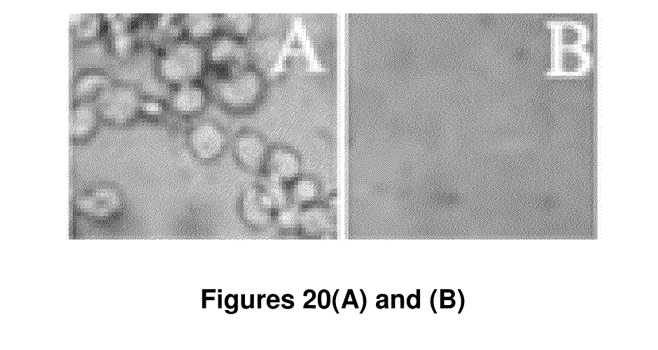

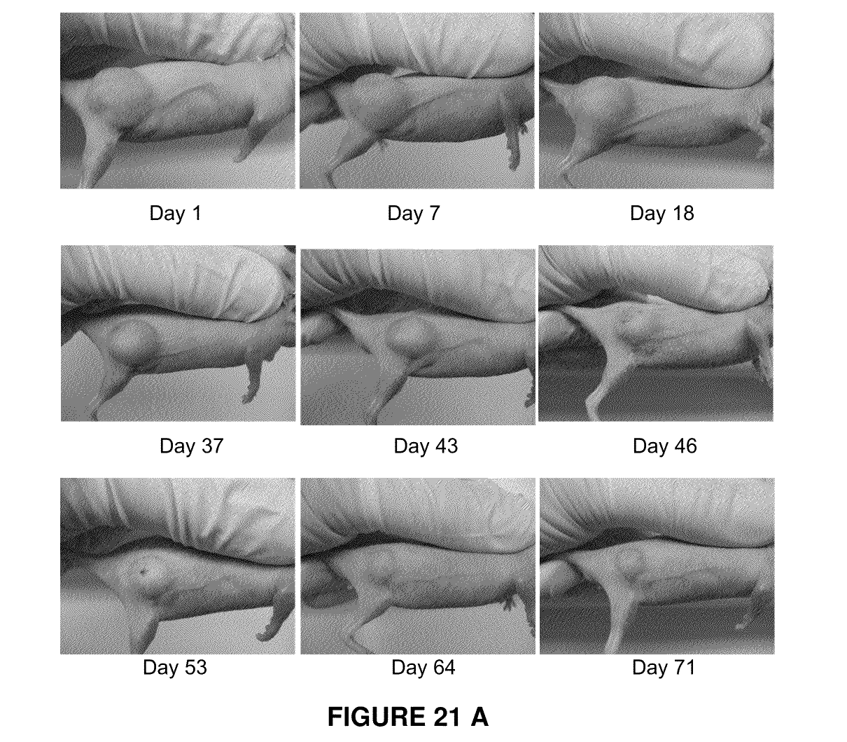

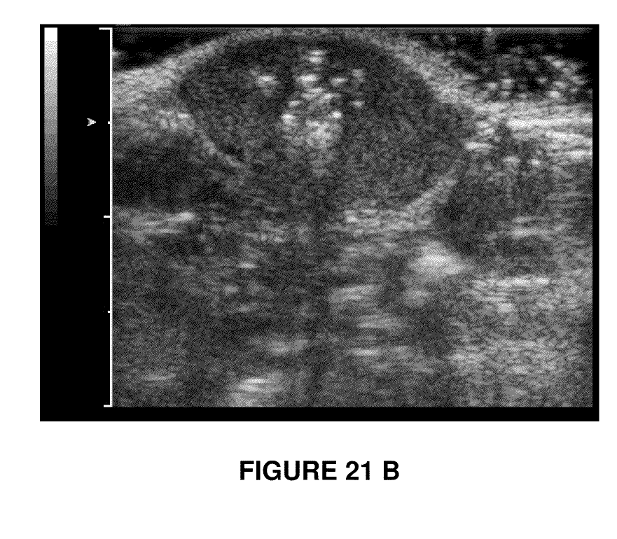

The nanoemulsions described herein can also include pluronics. An example of pluronics includes, but is not limited to, Pluronic L-61, pluronic 105, pluronic 85 or any combination thereof. Pluronic L-61 has been used in a SP1049C (micellar doxorubicin formulation) as a sensitizer of multidrug resistant cells (V. Alakhov et al., Block copolymer based formulations of doxorubicin. From cell screen to clinical trials. Colloids and Surfaces B: Biointerfaces 16 (1999) 113-134). In this aspect, by incorporating pluronics within the nanoemulsions, the nanoemulsion's sensitivity to low-frequency ultrasonic radiation may increase due to an increase in nanodroplet size; cancer cell sensitivity may increase due to enhanced sensitivity to hyperthermia (See FIG. 20), due to suppression of multidrug resistance, and due to more efficient delivery of therapeutic agents to the tumor may take place (see FIG. 21A, B). In a further aspect, as stated above, the pluronics may further function as a MDR suppressing agent. In some aspects, pluronics such as Pluronic L-61 can be incorporated into the nanoemulsion and act to enhance a tumor's hyperthermia sensitivity. In some aspects, heat or hyperthermia can be administered to the tumor concurrently with either ultrasonic radiation step described above or after either ultrasonic radiation step. In some aspects, hyperthermia is only administered to the tumor either concurrently with the second ultrasonic radiation step or after the second ultrasonic radiation step. As shown in FIGS. 20 and 21, tumors that were contacted with nanoemulsions having Pluronic L-61 and subsequently exposed to mild hyperthermia completely suppressed GEM resistance of the cells. A suppression of cell proliferation was observed also in the absence of GEM indicating that the main effect of Pluronic L-61 was related to cell sensitization to hyperthermia.

The nanoemulsions (i.e., the micelles and nanodroplets) can be prepared by using, for example, a solvent exchange technique. In some aspects, the nanoemulsions can include at least one therapeutic agent and a block copolymer, which are dissolved into solution and mixed with any one of the following: dimethyl sulfoxide (DMSO), tetrahydrofuran (THF), or dioxane. In some aspects, the therapeutic agent can be from 0 wt % to 25 wt %. In some aspects, the block copolymer can be from 0.1 wt % to 7 wt %. In some aspects, the copolymer can be from 0.25 wt % to 2 wt %. In some aspects, when preparing the nanodroplets, a halogen containing compound as described above can be added and mixed either by vortexing or using sonication with the solution that contains the at least one therapeutic agent and block copolymer. For example, a halogen containing compound (i.e., a perfluorocarbon) can be added to the solution containing at least one therapeutic agent mixed with the block copolymer and sonicated in ice-cold water by using 20 kHz to 3 MHz ultrasonic radiation. In some aspects, the halogen containing compound is 0.1 wt % to 10 wt % of total solution. In some aspects, the halogen containing compound is 0.5 wt % to 2 wt %.

In some aspects, the first nanoemulsion and the second nanoemulsion described herein can independently include a polyethylene glycol and a second polymer that form the block copolymer, a perfluorocarbon as described above, a therapeutic agent, or any combination thereof. In some aspects, the nanoemulsion described herein can include a polyethylene glycol and a second polymer that form the block copolymer and a therapeutic agent. In some aspects, the nanoemulsions described herein can include a polyethylene glycol and a second polymer that form the block copolymer, a perfluorocarbon, and a therapeutic agent. In some aspects, the nanoemulsions described herein can be mixtures of (1) a polyethylene glycol and a second polymer that form the block copolymer and a therapeutic agent and (2) a polyethylene glycol and a second polymer that form the block copolymer, a perfluorocarbon, and a therapeutic agent.

For example, in some aspects, the nanoemulsions described herein can include a polyethylene glycol poly(l)lactic acid block copolymer, a polyethylene glycol poly(d,l)lactic acid block, a perfluoropentane, a therapeutic agent, or any combination thereof. In some aspects, the nanoemulsion described herein can include a polyethylene glycol poly(l)lactic acid block copolymer and a therapeutic agent. In some aspects, the nanoemulsions described herein can include a polyethylene glycol poly(l)lactic acid block copolymer, a perfluoropentane, and a therapeutic agent. In some aspects, the nanoemulsions described herein can be mixtures of (1) a polyethylene glycol poly(l)lactic acid block copolymer and a therapeutic agent and (2) a polyethylene glycol poly(l)lactic acid block copolymer, a perfluoropentane, and a therapeutic agent.

In some aspects, the nanoemulsions can include a polyethylene glycol polycaprolactone block copolymer, a perfluoropentane, a therapeutic agent or any combination thereof. For example, in some aspects, the nanoemulsion can include a polyethylene glycol polycaprolactone block copolymer and a therapeutic agent. In some aspects, the nanoemulsions can include a polyethylene glycol polycaprolactone block copolymer, a perfluoropentane, and a therapeutic agent. In some aspects, the nanoemulsions described herein can be mixtures of (1) a polyethylene glycol polycaprolactone block copolymer and a therapeutic agent and (2) a polyethylene glycol polycaprolactone block copolymer, a perfluoropentane, and a therapeutic agent.

In some aspects, the nanoemulsions can include mixtures of (1) a polyethylene glycol polycaprolactone block copolymer, a perfluoropentane, a therapeutic agent or any combination thereof; (2) polyethylene glycol poly(l)lactic acid block copolymer, a perfluoropentane, a therapeutic agent or any combination thereof; (3) polyethylene glycol poly(d,l)lactic acid block copolymer, a perfluoropentane, a therapeutic agent or any combination thereof; or (4) any combination thereof.

In a further aspect, the nanoemulsions include a polyethylene glycol poly(l)lactic acid block copolymer, a perfluoropentane, and a therapeutic agent, wherein the therapeutic agent comprises paclitaxel, doxorubicin, gemcitabine, or any combination thereof. In some aspects, the nanoemulsion includes a polyethylene glycol polycaprolactone block copolymer, a perfluoropentane, and a therapeutic agent, wherein the therapeutic agent comprises paclitaxel, doxorubicin, gemcitabine, or any combination thereof.

In some aspects, the stability, drug loading capacity, and ultrasound sensitivity of nanoemulsions depend on a number of factors including but not limited to a type of a block copolymer, stereospecificity of blocks in a block copolymer, and block lengths in a block copolymer. More stable nanodroplets are more beneficial for drug carrying because they prevent premature drug release. However, in some aspects, stronger nandroplet walls make these nanodroplets less susceptible to ultrasound. The methods described herein increase susceptibility of nanodroplets to the action of ultrasound, thus enhancing droplet-to-bubble conversion and drug release from nanodroplets with strong walls. For example, nanodroplets with strong walls can include nanodroplets stabilized by polyethylene glycol poly (l)lactic acid.

The methods described herein can be used to treat subjects having cancer (e.g, a subject with a malignant or benign tumor). As described above, these tumors can include, but are not limited to breast tumors, ovarian tumors, pancreatic tumors, or a combination thereof. In one aspect, the method of treating a tumor in a subject can include the following steps:

(a) injecting a therapeutic agent encapsulated in a first nanoemulsion into the subject;

(b) exposing the tumor to a first ultrasonic radiation of less than 300 kHz; and

(c) exposing the tumor to a second ultrasonic radiation from about 1 MHz to about 5 MHz to the tumor.

In this aspect, the first nanoemulsion can be injected intravenously, subcutaneously, intramuscularly, intratumorally, or any combination thereof. In some aspects, the first nanoemulsion is only injected intravenously (i.e., systemic administration). In a further aspect, a second nanoemulsion can be directly injected into the tumor via intratumoral injection prior to step (b). In yet another aspect, the second nanoemulsion can be directly injected into the tumor before step (a) but prior to step (b).

In some aspects, the method of treating a tumor in a subject can be repeated. For example, the subject can be injected with the nanoemulsion twice a week for two or three consecutive weeks. After each injection, the subject can be exposed to ultrasonic radiation at an optimal time, which is determined based on the type and pharmacokinetics of nanoemulsion inject. After this two week period, the subject does not receive any treatment for the next two weeks. After this two week lapse or break in treatment, the subject can again be injected with the nanoemulsion twice a week for two or three consecutive weeks. As stated above, after each injection, the subject can be exposed to ultrasonic radiation at an optimal time, which is determined based on the type and on the pharmacokinetics of the nanoemulsion injected. For further detail refer to the examples section and to FIG. 20. In another aspect, the treatment may be given twice a week for four consecutive weeks without interruption.

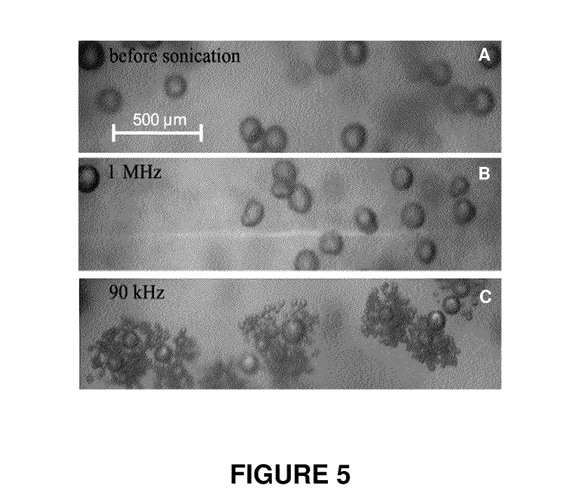

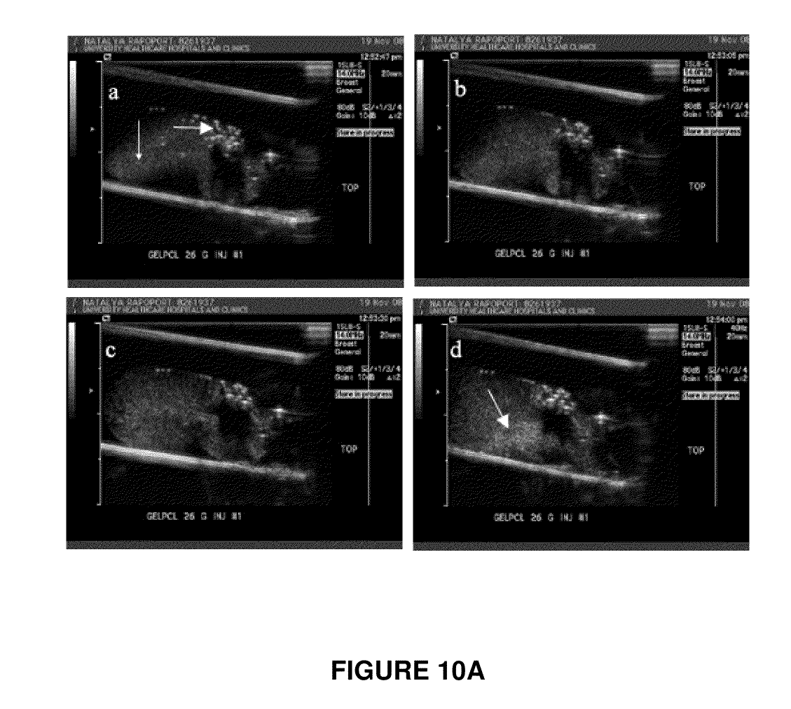

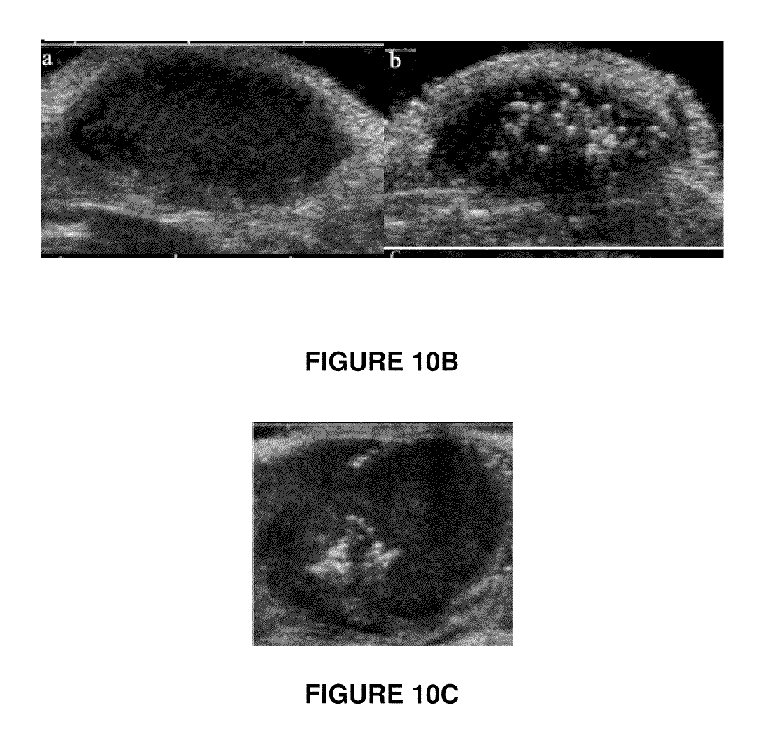

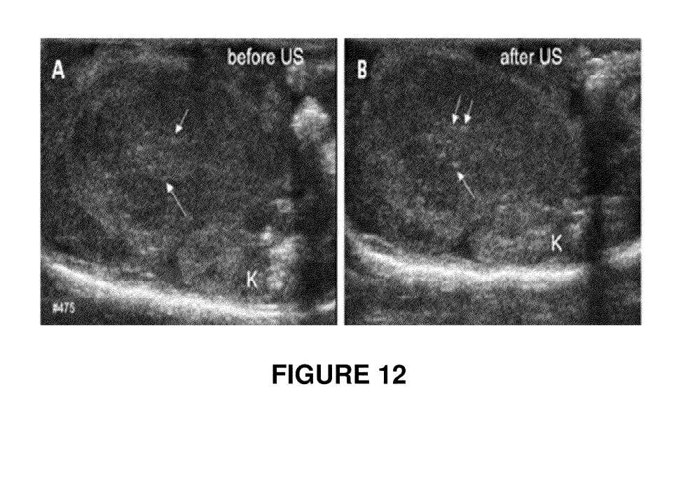

In some aspects, the method of treating a tumor in a subject, in which the subject can include a human, can be as follows: Weekly treatment with paclitaxel dose ranging from 30 mg/m² to 135 mg/m² depending on the tumor type and localization, followed by electronic or mechanical steering of a focused ultrasound beam over a tumor, with each sonicated volume being exposed to ultrasound for a desired time ranging from seconds to minutes. In some aspects, the tumor is directly injected with a second nanoemulsion prior to the first ultrasonic radiation step; the second nanoemulsion may or may not include a therapeutic agent. In some aspects, this step generates microbubbles in tumor tissue (see FIG. 10 A-C). Though low-frequency ultrasound does not allow sharp focusing, it is beneficial for large and/or deeply located tumors due to deep penetration into a body. A second ultrasonic radiation step with at least 1-MHz or higher frequency can be performed with high precision. Due to a long preservation of microbubbles formed in the tumor tissue during the direct injection of nanoemulsion (see FIG. 21 B) and their catalytic action on droplet-to-bubble transition (see FIGS. 5 and 12), the direct nanoemulsion injection step is not required with every systemic injection. Direct intratumoral injection may be performed every three to four weeks.

If desired, the first ultrasonic radiation can be less than about 200 kHz, less than 100 kHz, less than 95 kHz, less than or equal to 90 kHz, less than or equal to 80 kHz, less than or equal to 70 kHz, less than or equal to 60 kHz, less than or equal to 50 kHz, less than or equal to 40 kHz, less than or equal to 30 kHz, or equal to 20 kHz. If desired, the second ultrasonic radiation can range from about 1 MHz to about 4 MHz, from about 1 MHz to about 3.5 MHz, from about 1 MHz to about 3.0 MHz, or from about 1 MHz to about 2.5 MHz. In each of these aspects, the nanoemulsion, which contains an encapsulated therapeutic agent, will undergo acoustic droplet vaporization (ADV) and form microbubbles within the tumor. In this aspect, the therapeutic agent will be efficiently and effectively administered to the tumor and will reduce tumor size and prevent tumor cell proliferation. In some aspects, Pluronics such as Pluronic L-61 can be further incorporated into the nanoemulsion and act to enhance a tumor's hyperthermia sensitivity. In some aspects, Pluronic L-61 can be incorporated into either the first nanoemulsion which can include a drug loaded nanoemulsion of the systemic injection or either the second nanoemulsion which can be an empty or drug loaded nanoemulsion of the direct intratumoral injection. In some aspects, heat or hyperthermia can be administered to the tumor concurrently with either ultrasonic radiation steps described above or after either ultrasonic radiation steps. In some aspects, ultrasound or ultrasonic radiation can be used to generate desired hyperthermic conditions. In some aspects, hyperthermia is only administered to the tumor either concurrently with the second ultrasonic radiation step or after the second ultrasonic radiation step. In certain aspects, the methods described herein will kill the tumor without damaging the surrounding normal cells and tissues.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

### Block Copolymers

Block copolymers used in this study were from Polymer Source Inc. (Montreal, Quebec, Canada). The polyethylene glycol poly(l)lactic acid (PEG-PLLA) copolymer had a total molecular weight of 9,700; the molecular weights of a hydrophilic PEG block and a hydrophobic PLLA block were 5,000 D and 4,700 D respectively. The number of the monomer units in the corresponding blocks was 113.6 and 54.7. The polyethylene glycol polycaprolactone (PEG-PCL) copolymer had a total molecular weight of 4,600 D; the molecular weights of a PEG block and PCL block were 2000 D and 2600 D respectively. The number of monomer units in the corresponding blocks was 45.5 and 22.8.

### Micellar Solutions and Drug Loading

Micellar solutions of the PEG-PLLA and PEG-PCL block copolymers were prepared by a solvent exchange technique as described in detail previously. DOX loading into micelles was performed at the micelle preparation stage. PTX encapsulated in methoxy PEG-poly(D, L-lactide) micelles, Genexol-PM (GEN), was from Samyang Corp. A desired weight of the GEN powder was dissolved in the PEG-PLLA or PEG-PCL micellar solution.

### Formulations

PTX-loaded nanoemulsions were prepared as follows: micellar-encapsulated PTX (GEN) was dissolved in 0.25% PEG-PLLA micelles; 1% vol. perfluoropentane (PFP) was added to this solution and samples were sonicated in ice-cold water by 20-kHz ultrasound ultrasound (VCX500, Sonics & materials Inc., Newtown, Conn., USA) until all PFP was transferred into an emulsion. In what follows, this formulation is called nbGEN.

DOX-loaded nanoemulsions were prepared as previously described. Briefly, the drug was first loaded into the PEG-PLLA or PEG-PCL micelles. PFP was added to this micellar solution and the mixture was sonicated as described above.

Nanodroplet Introduction into Gels

The nanodroplets were mixed with 0.2% agarose solution in PBS at 35° C. The liquid mixture was placed in a Samco transfer pipette (5-mm inner diameter, 0.3-mm wall thickness) (Fisher Scientific, Pittsburg, Pa., USA) and cooled down to room temperature for gel formation. For the droplet introduction into the bovine plasma clots, equal volumes (200 μL each) of a nanodroplet emulsion and bovine plasma (Innovative Research, Novi, Mich., USA) were gently mixed. The clotting was initiated by adding 10 μL of 0.5 mol/L calcium chloride and 20 IU/mL bovine thrombin (Sigma-Aldrich, St. Louis, Mo., USA). The mixture was drawn into a Samco transfer pipette and incubated for 10 min at 37° C.

### Particle Size Distribution

Size distribution of nanoparticles was measured by dynamic light scattering at a scattering angle of 165° using Delsa Nano S instrument (Beckman Coulter, Osaka, Japan) equipped with a 658-nm laser and a temperature controller. Particle size distribution was analyzed using the non-negative least squares (NNLS) method.

The instrument allows measurement of particle sizes from 0.6 nm to 7 μm; microparticles larger than 7 μm cannot be measured accurately. Optical monitoring of the samples using an inverted microscope and hemacytometer (model 3200, Hauser Scientific, Horsham, Pa., USA) showed no microdroplets larger than 7 μm. The hemacytometer was used for measuring the mean concentration of microdroplets.

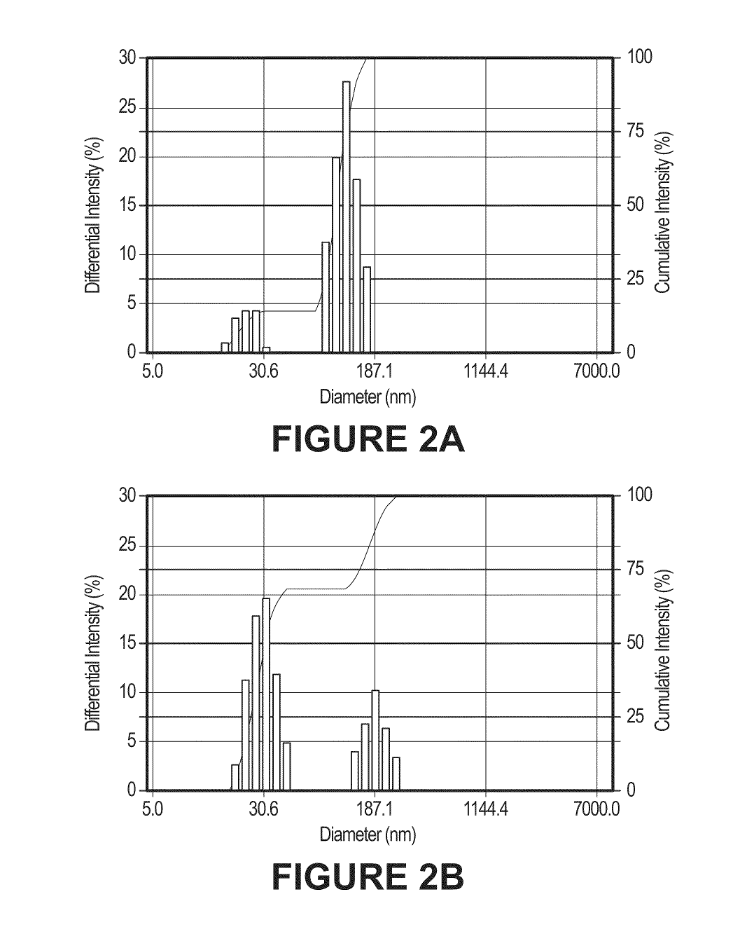

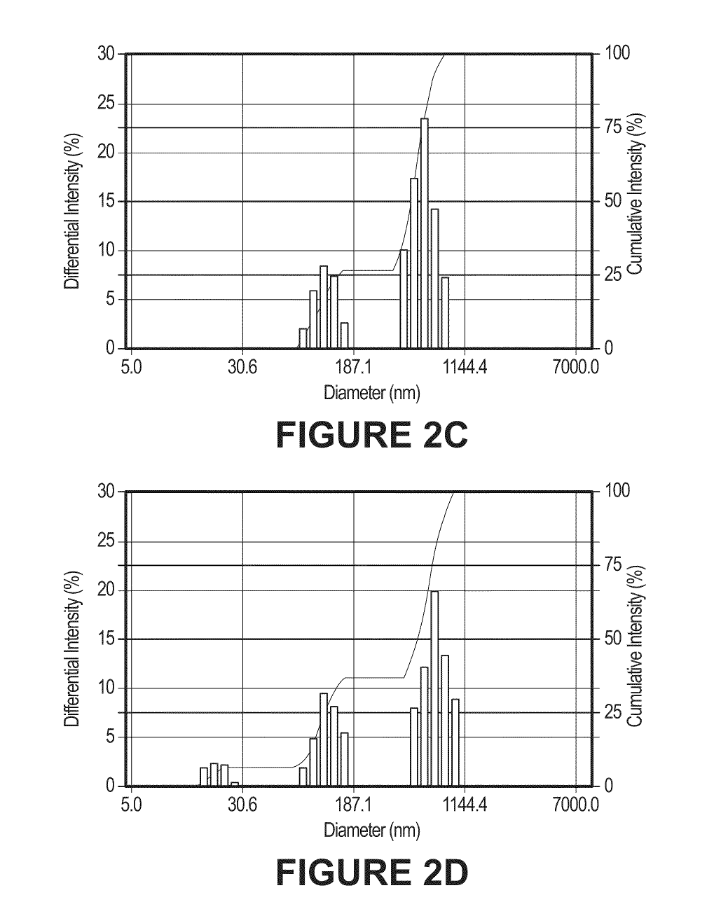

The size distribution of the nanoparticles was typically bimodal, as shown in FIG. 2. The corresponding distribution parameters (diameter at the peak of the distribution and volume fraction of a corresponding population) are presented in Table 1. The smaller particles corresponded presumably to individual spherical micelles while larger particles represented either worm-like micelles or micellar aggregates. For both types of particles, paclitaxel-loaded micelles (29.3 nm) were larger than empty micelles (22.2 nm). The formation of nanoemulsion after the perfluoropentane (PFP) introduction resulted in the disappearance of small micelles and generation of nanodroplets (592.6 nm, 73%). For paclitaxel-loaded systems, introduction of PFP resulted in a tri-modal size distribution. The size of micelles dropped from 29.3 nm to 19.3 nm while the size of nanodroplets increased from 592.6 to 718.4 nm, suggesting paclitaxel transfer from micelles to nanodroplets. Based on this information, in the formulation used in vivo, the entirety of the drug may be considered located in nanodroplets.

TABLE 1
Size distribution parameters of micellar and nanoemulsion systems at
room temperature.

| | Peak 1 | | Peak 2 | | Peak 3 | |
|---|---|---|---|---|---|---|
| | | Volume | Radius, | Volume | Radius, | Volume |
| Samples | Radius, nm | fraction | nm | fraction | nm | fraction |
| 0.25% PEG-PLLA | 22.2 | 15% | 114.7 | 85% | — | — |
| micellar solution | | | | | | |
| GEN in 0.25% PEG- | 29.3 | 69% | 188.9 | 31% | — | — |
| PLLA micellar solution | | | | | | |
| 1% PFP/0.25% PEG- | — | — | 117.5 | 27% | 592.6 | 73% |
| PLLA emulsion | | | | | | |
| GEN in 1% PFP/0.25% | 19.3 | 7% | 122.9 | 31% | 718.4 | 62% |
| PEG-PLLA emulsion | | | | | | |

### Sonication

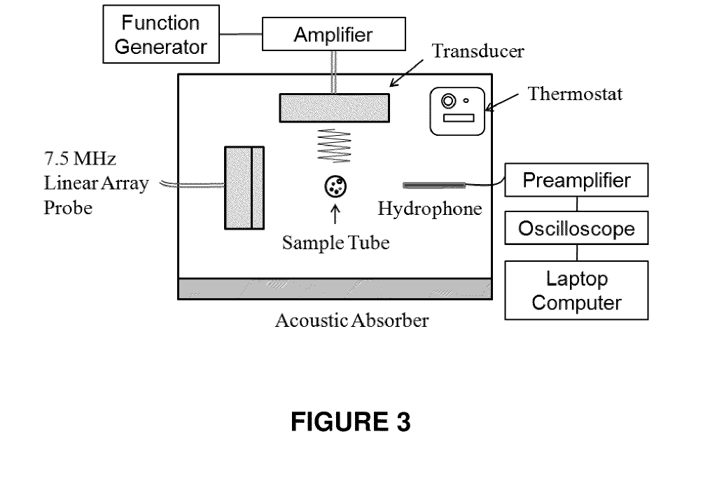

Unfocused 1-MHz ultrasound was generated by an Omnisound 3000 instrument (Accelerated Care Plus Inc, Sparks, Nev., USA) equipped with a 1-cm² piezoceramic crystal and 5-cm² probe head. Focused 1 MHz ultrasound was generated by a high intensity focused ultrasound (HIFU) transducer (H-101, Sonic Concepts, Bothell, Wash., USA) with an active diameter of 64 mm and focal length of 63 mm. The −3 dB lateral and axial pressure profiles were 1.2 and 10 mm respectively. Transducer was driven by an arbitrary waveform generator (33120A, Agilent, Santa Clara, Calif., USA) connected to a 50-dB RF power amplifier (Model 240L, Electronics Navigation Industries, Rochester, N.Y., USA). The nanodroplet formulation, drawn into a Samco polyethylene transfer pipette (5-mm inner diameter, 0.3-mm wall thickness) (Fisher Scientific Pittsburgh, Pa., USA) was positioned either at a distance of 0.5 cm from the unfocusing transducer or at the focal zone of the focusing transducer (See the scheme and the photograph, FIG. 3). The focal point of a HIFU transducer was detected using an xyz-positioner and hydrophone. The arrangement was housed in an open glass tank containing filtered distilled degassed water maintained at room temperature or 37° C. using a temperature controller (Polystat, Cole-Parmer, Vernon Hills, Ill., USA). To minimize possible standing wave formation, an absorbing rubber liner was mounted opposite the transducer.

Ninety kilohertz ultrasound was generated in the SC-100 ultrasound bath (Sonicor Instrument Co., Copiague, N.Y., USA).

### Sound Attenuation

Sound phase velocity was measured by a single-sample technique. The sample was injected in the Samco transfer pipette (Fisher Scientific, Pittsburgh, Pa.) of a 12.5 mm internal diameter and placed on the way of the ultrasound beam that was generated by pulsed transmitting transducer having a central frequency of 500 kHz (V318, Panametrics, Waltham, Mass., USA). On the other side of the sample the beam was peaked up by hydrophone (TNU 100A, NTR Systems, Seattle, Wash., USA) with a 40 dB preamplifier (Model 5678, Panametrics, Waltham, Mass., USA). A time-domain signal of the received pressure waveform was stored for further analysis. The data were analyzed using MATLAB software (The MathWorks, Natick, Mass., USA). At each given ultrasound frequency, the relative phase velocity and attenuation coefficient of a sample were calculated by comparing with the waveform from phosphate buffered saline (PBS). Attenuation was calculated using the following equation; because both sample and PBS were measured in the same pipette, no correction for reflection from the pipette wall was introduced.

$$Attenuation = \frac{[ 10 {log}_{10} ( {\sum }^{n} {p}_{PBS}^{2} / {\sum }^{n} {p}^{2} ) ]}{L} \qquad ( 1 )$$

where n is the number of data points, p and p_{PBS} is pressure of a sound wave in the sample and PBS respectively.

### Monitoring of Acoustic Droplet Vaporization by Visual Observation and Ultrasound Imaging

The ultrasound-induced formation of microbubbles from nanodroplets was monitored at room temperature visually and by ultrasound imaging, based on a higher echogenicity of bubbles compared to droplets (Kripfgans, Fowlkes 2000, Lo, Kripfgans 2007); a 7.5-MHz linear array scanner (Scanner 250, Pie Medical, Maastricht, The Netherlands) was used for ultrasound imaging with 14 frames per second scan rate. The samples in Samco transfer pipettes (5-mm inner diameter, 0.3-mm wall thickness, 2 mm diameter of the narrow bottom part) were allowed to precipitate overnight to the bottom of the transfer pipette. The samples were then sonicated by 1-MHz ultrasound starting with the lowest pressure of 0.14 MPa generated by the Omnisound 3000 instrument at the site of the sample. The pressure was increased stepwise and the formation of the upward directed bubble stream was monitored. The lowest pressure that induced bubble stream formation was considered corresponding to or being above the ADV threshold for the formation of primary bubbles. Due to a stepwise nature of the pressure increase, we can only state that the ADV threshold for a particular sample was located in the interval between the highest pressure that did not induce bubble formation and the next step pressure that induced vaporization.

By assuming a 125-fold density difference between the PFP in the liquid and gaseous phases, we estimated that the density of a droplet will be equal to the water density when the degree of vaporization inside an individual droplet equals 40%. At a higher degree of vaporization, droplets will rise in water. At each ultrasound power, the ultrasound was turned off to monitor if bubbles precipitated to the bottom of the test tube due to reversibility of vaporization or were rising to the sample surface due to the irreversible formation of stable bubbles. The resolution of the ultrasound images was lower than 200 μm, thus bubbles observed by imaging were most probably secondary bubbles formed via coalescence of primary bubbles. Still, visual observation of the initial bubble stream formation and ultrasound imaging produced close results on the ADV thresholds suggesting that ADV was a limiting step for bubble coalescence (i.e. bubble coalescence occurred faster than droplet vaporization). The results were reproducible in parallel runs.

### Cavitation Activity

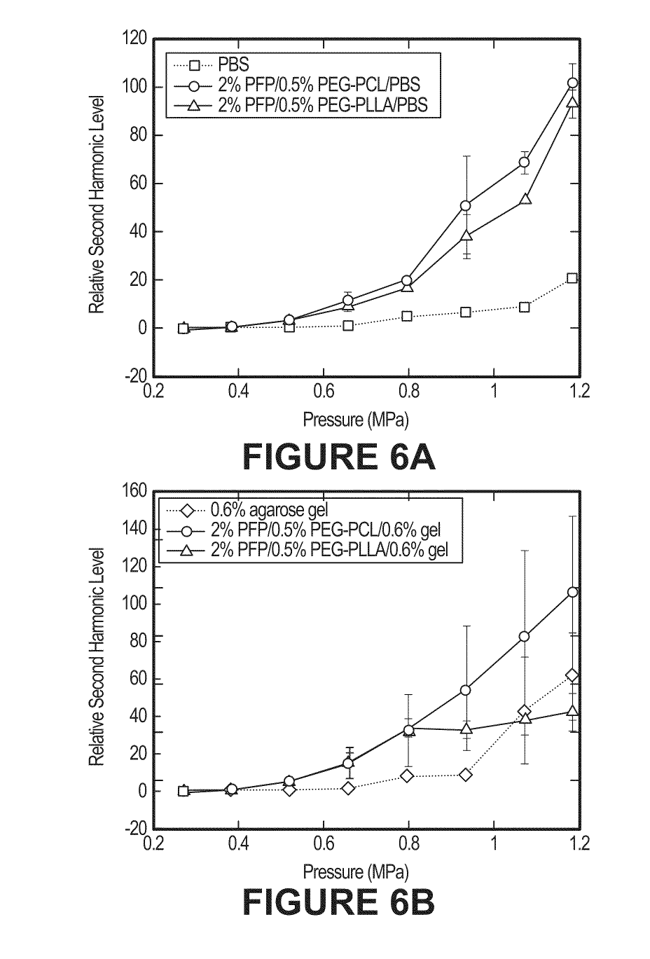

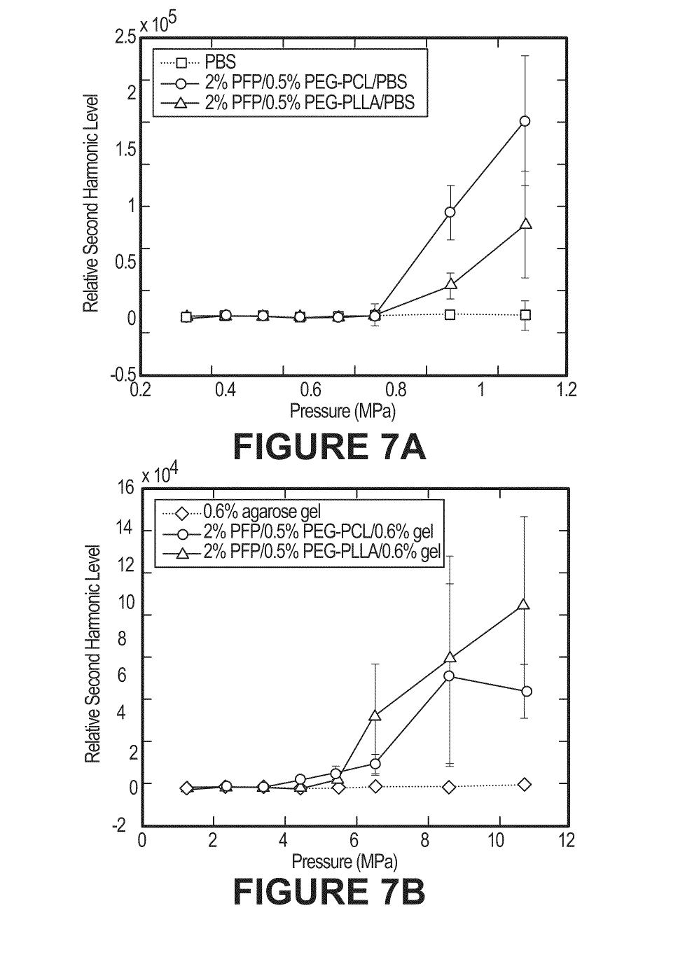

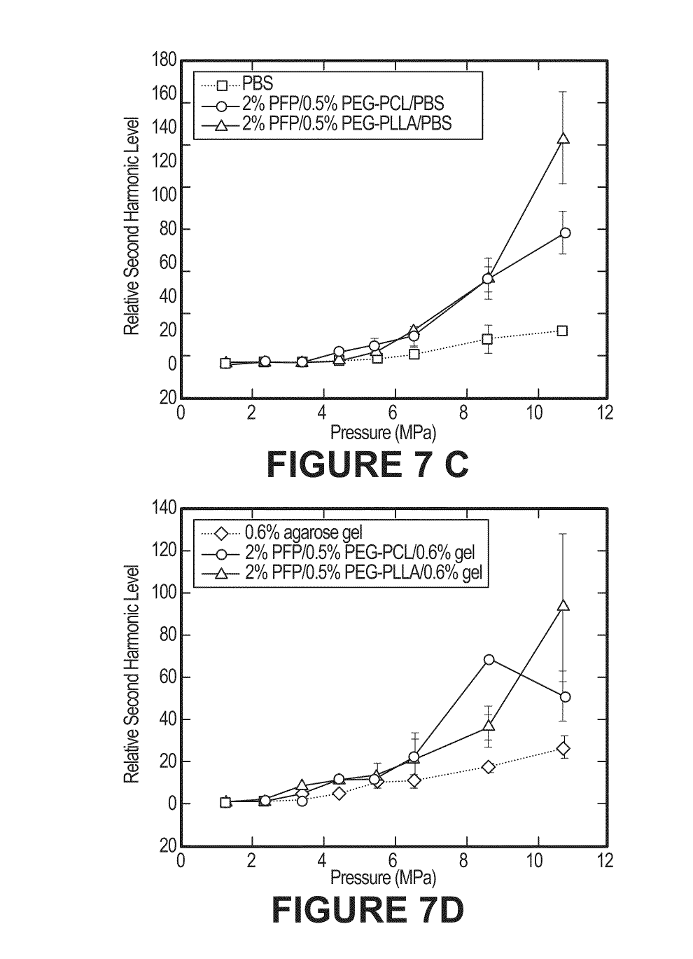

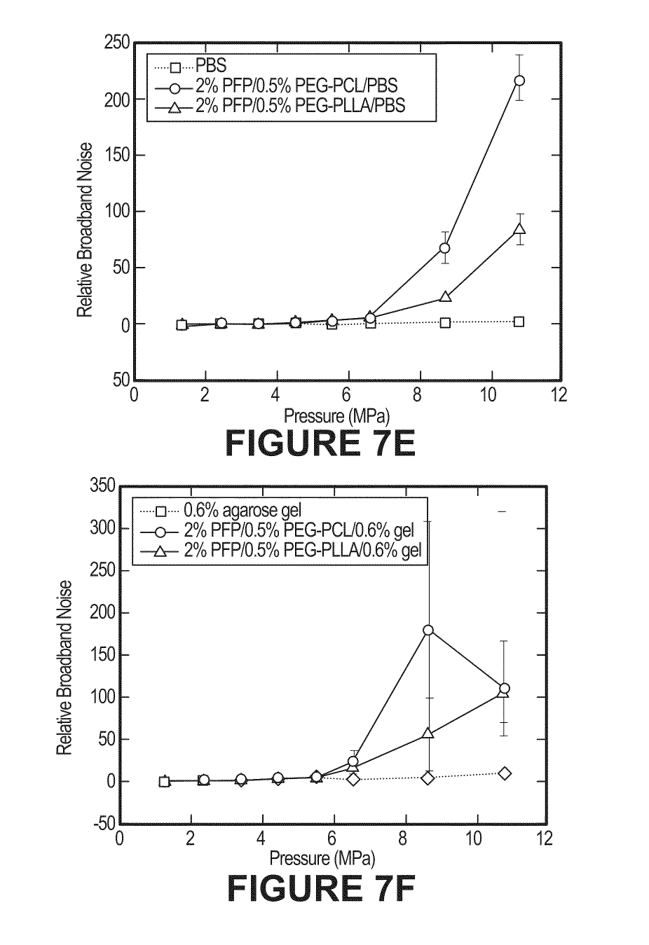

The measurements were performed with the samples placed in the transfer pipettes. Before placement into the transfer pipette, the samples were carefully pumped in and out in order to lift the precipitated droplet population and mix the sample. To eliminate bubbles that could be formed during the sample transfer into the transfer pipette, the cavitation measurements started five minutes after sample was inserted in the experimental setup; sound attenuation measurements indicated that this time interval was sufficient for bubble elimination. Cavitation activity was assessed by measuring subharmonic and broadband noise amplitudes in a portion of the scattered beam. To detect the acoustic emissions from cavitation, a needle hydrophone (HNR-0500, Onda, Sunnyvale, Calif., USA) with a 20-dB preamplifier (AH-1100, Onda, Sunnyvale, Calif., USA) was mounted at a 90° angle to the transducer (See FIG. 3); the vertical position of the hydrophone corresponded to the center of the sample holder. The radiofrequency (RF) signals were digitized with a sampling frequency of 500 MHz with an oscilloscope (TDS 3012B, Tektronix, Beaverton, Oreg., USA). For ten seconds, a total of 15 recordings of time-domain RF signals emitted from the droplets samples were acquired for each ultrasound pressure level and stored on a laptop computer for further analysis. The temporal waveform was gated with a 40 μs Hamming window and the fast Fourier transform (FFT) was computed to determine frequency content. Broadband noise was quantified as the root-mean-square (RMS) over a selected combination of frequency bands (0.6-0.9 MHz, 1.1-1.4 MHz, 1.6-1.9 MHz, 2.1-2.4 MHz, and 2.6-2.9 MHz) to isolate the broadband emission from the fundamental, harmonic, and ultraharmonic frequencies. The amplitudes of a subharmonic component were taken from the peaks in the FFT spectrum at the half of the fundamental frequency. The relative level (RL) of the broadband noise and the subharmonic component was each calculated according to the following equation:

$$RL = \frac{{L}_{S} - {L}_{B}}{{L}_{B}} \qquad ( 2 )$$

where the L_{S} and L_{B} indicate the amplitude levels from a sample and from water, respectively; the amplitude for water was taken at the lowest insonation pressure as a noncavitating baseline. A total of 75 FFT spectra from five measurements at each insonation pressure were averaged and presented in FIGS. 6 and 7. The pressure levels presented in this application are peak-to-peak pressures. All data were processed using Matlab software. The cavitation threshold was considered to correspond to the ultrasound pressure at which the separation between the experimental point and a reference point (in our case PBS in the transfer pipette) was larger than three standard deviations for the reference point.

### Cells

Ovarian cancer A2780 cells were obtained from American Type Culture Collection (Manassas, Va., USA). The cells were cultured in RPMI-1640 medium supplemented with 10% FBS at 37° C. in humidified air containing 5% CO₂.

For experiments involving pancreatic tumors, pancreatic cancer MiaPaCa-2 cells were obtained from American Type Culture Collection (Manassas, Va.). Cells were maintained in DMEM supplemented with 10% heat-inactivated fetal bovine serum (FBS). MiaPaCa-2 cells were transfected with red fluorescence protein (RFP) using a previously described procedure. Cells were cultured at 37° C. in humidified air containing 5% CO₂.

### Animal Procedures: Ovarian Cancer Model

The 4- to 6-week old nu/nu mice from Charles River Laboratories (Wilmington, Mass., USA) were used to monitor the effect of the intravenously injected nanodroplet-encapsulated PTX or DOX and ultrasound on tumor growth. Animals were housed in accordance with the Guide for the Care and Use of Laboratory Animals as adopted by the National Institutes of Health. All experiments were performed in accordance with the guidelines of the Institutional Animal Care and Use Committee of the University of Utah (Protocol 08-01001). For inoculation, ovarian carcinoma A2780 cells were suspended in 100 μL serum-free RPMI-1640 medium and inoculated subcutaneously to the flanks of unanaesthetized mice (1×10⁶ cells per mouse).

In the pilot experiments with ovarian cancer model, mice were randomly assigned to four groups: (1) negative control was used to monitor tumor growth rate in untreated animals (N=3); (2) treatment by systemic injections of 1% PFP/0.25% PEG-PLLA nanoemulsion formulation of PTX, nbGEN (20 mg/kg as PTX) combined with ultrasound (N=3) (3) one mouse was inoculated with two tumors in the right and left flank was treated by systemic injections of nbGEN (20 mg/kg as PTX) given twice weekly for two weeks. Only one—the right tumor was sonicated; (4) one mouse was treated by the empty (i.e. not drug-loaded) 1% PFP/0.25% PEG-PLLA nanoemulsion and ultrasound. Ultrasound treated groups were sonicated by 1-MHz continuous wave (CW) ultrasound at 3.4 W/cm² nominal power density for 60 s; ultrasound was applied four to five hours after the drug injection through a water bag coupled to a mouse skin by the ultrasound coupling gel.

Injected volume in all cases was 200 μL. The upper limit of the injected nanodroplet dose was estimated based on the concentration of the introduced PFP and the measured nanodroplet size distribution according to the following equation:

$${N}_{m} = \frac{v \times f \times {10}^{9}}{4 π {r}^{3} / 3} \qquad ( 3 )$$

where v=2 μL is the volume of PFP in 200 μL of the injected nanoemulsion, f is a volume fraction of a selected droplet population, and r is the droplet radius in micrometers at the peak of a selected population (in this estimation, the loss of the PFP at the sample preparation and the thickness of the droplet shell were neglected). The estimated upper limit of the injected nanodroplet dose was about 7×10⁹ per mouse for nanodroplets with a peak diameter of 0.7 μm.

Tumor volume (V) was calculated as follows:

V=L×W²/2 (4)

where L and W are the length and the width of the tumor, respectively.

The normalized tumor size (Vₙ) was calculated from the initial tumor volume (V₀) according to the following formula:

$${V}_{n} = \sqrt[3]{\frac{V}{{V}_{0}}} \qquad ( 5 )$$

### Animal Procedures: Pancreatic Cancer Model

Orthotopic pancreatic cancer was inoculated surgically in the pancreatic tail. Mice received a single sub-capsular injection of 1×10⁶ red fluorescent protein labeled MiaPaCa-2 cells suspended in 0.125 mL serum free media (DMEM). All procedures were done utilizing a 12× Universal S3B microscope.

Following the primary surgery, high resolution (3456 pixels×2304 pixels) whole body digital images (EOS Digital Rebel, Canon USA, Lake Success, N.Y.) of each mouse were obtained once a week to monitor primary tumor growth and presence of metastases. The red fluorescent protein was visualized with an Illumatool Bright Light System that consisted of a 563 nm excitation filter and a 587 nm emission filter (Model LT-9900, LightTools Research, Encinitas, Calif.). Animals were imaged under nose-cone induced isoflurane general anesthesia. Primary tumor area was quantified using public domain software ImageJ (National Institutes of Health http://rsb.info.nih.gov/ij/).

Animals were randomly assigned to six groups, six animals each: (1) negative control (injection of PBS); (2) GEM at 140 mg/kg (positive control 1); (3) GEN at 20 mg/kg as PTX (positive control 2); (4) GEM+GEN combination treatment; (5) GEN+ultrasound; (6) GEM+nbGEN+ultrasound. Unfocused continuous wave 1-MHz ultrasound was applied extracorporeally for 30 s to the pancreas region of abdomen. Nominal ultrasound intensity was 3.4 W/cm², which corresponded to a measured MI=0.59. Ultrasound was applied extracorporeally to abdominal area in the pancreas region through a water bag coupled from both sides to ultrasound transducer and mouse skin by the ultrasound coupling gel. Ultrasound was applied 4 to 5 hours after drug injection. In the first treatment round, drug was injected twice a week for two weeks then there was a break for two weeks and the treatment was repeated using the same protocol as in the first treatment round. The dose of PTX was the same in all formulation used in this study.

### Results

### Systemic Chemotherapy of a Mouse Bearing Two Ovarian Carcinoma Tumors: Effect of Ultrasound

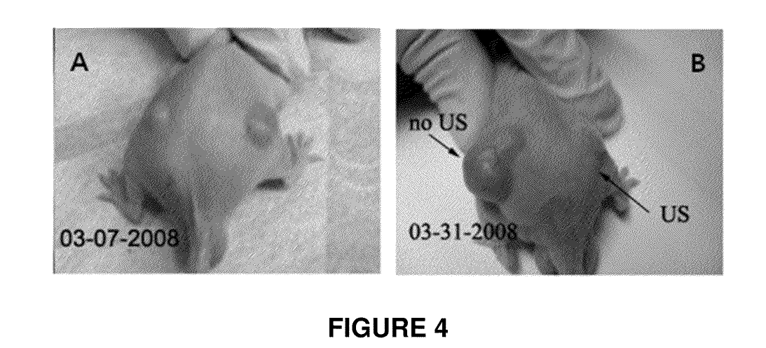

The results of chemotherapy of the mouse bearing two ovarian carcinoma tumors inoculated in the right and left flank are presented in FIG. 4. This mouse was treated by four systemic injections of nbGEN (20 mg/kg as PTX) given twice weekly; only one (the right) tumor was sonicated. The unsonicated left tumor grew with the same rate as control tumors (for which growth rates were measured separately). In contrast, the sonicated tumor appeared completely resolved after four treatments. These data indicated that without ultrasound, PTX was tightly retained by the nanodroplet carrier in vivo, which provides protection of healthy tissues. However, PTX was effectively released into the tumor volume under the action of ultrasound, which resulted in efficient tumor regression.

### Acoustic Droplet Vaporization

The goal of these experiments was to examine conditions of the droplet-to-bubble transition in liquid systems and gels. Nanodroplet vaporization to generate bubbles is highly desirable for both ultrasonography and drug delivery. Due to the high acoustic impedance, perfluorocarbon droplets manifest echogenic properties; however, bubbles show much higher echogenicity than droplets, which is important for in vivo ultrasound imaging. Besides producing high ultrasound contrast, cavitating bubbles serve as potent enhancers of ultrasound-mediated drug delivery, which droplets do not offer.

Sonication of perfluorocarbon emulsions can induce droplet-to-bubble transition; this effect is called acoustic droplet vaporization, or ADV. In the present work, the ADV effect was studied for the block copolymer-stabilized perfluoropentane nanoemulsions used in the in vivo experiments.

The PFP has a boiling temperature of 29° C. at atmospheric pressure, thus producing nanoemulsions at room temperature but manifesting high propensity for vaporization at heating. However, for small droplets stabilized by elastic copolymer shells, the Laplace pressure may substantially increase boiling temperature. The Laplace pressure is the pressure difference between the inside and the outside of droplet or bubble. This effect is caused by the surface tension at the interface between bulk liquid and droplet liquid.

The Laplace pressure is given as

$$Δ P = {P}_{inside} - {P}_{outside} = \frac{2 σ}{r} \qquad ( 6 )$$

where P_{inside} is the pressure inside a droplet, P_{outside} is the pressure outside a droplet, σ is the surface tension, and r is droplet radius.

Excessive pressure inside a droplet results in increase of PFP boiling temperature. This phenomenon has important consequence for drug delivery. Because Laplace pressure is reversely proportional to droplet size according to eq. 6, smaller droplets have higher boiling temperatures than larger droplets.

Using the Antoine equation for the pressure dependence of vaporization temperature, vaporization temperatures of nanodroplets of various sizes were calculated for two surface tension values of σ=50 mN/m and σ=30 mN/m. These calculations showed that at physiological temperature of 37° C., the borderline droplet size is about 6.4 μm for σ=50 mN/m and about 4 μm for σ=30 mN/m. At 37° C., droplets smaller than 4 μm will remain in the liquid state while larger droplets will evaporate. However, droplets of these large sizes were not present in initial nanoemulsions (see Table 1). Therefore nanodroplets were expected to remain in circulation as liquid droplets rather than form microbubbles at physiological temperatures, which is beneficial for extravasation into tumor tissue. However after extravasation, droplet-to-bubble transition is desirable.

Three factors that induced droplet-to-bubble transition in block copolymer stabilized PFP nanodroplets were detected: a heating (thermal factor); a sonication (thermal and/or mechanical factor); and an injection through a thin needle (mechanical factor). Among these factors, ultrasound was the most powerful.

Ultrasound intensities that induced droplet-to-bubble transition were recorded. These experiments were performed for two types of the droplet-stabilizing copolymers (PEG-PLLA and PEG-PCL) with 1-MHz or 3-MHz ultrasound at room temperature and 37° C., in liquid emulsions and gels.

The data obtained for the liquid nanoemulsions may be relevant to the bubble behavior in circulation. However after extravasation into the tumor tissue, the droplets or bubbles are surrounded by a much more viscous extracellular matrix of the tumor interstitium. This situation was modeled by introducing the droplets into a 0.6% agarose gel or a plasma clot.

### ADV in Liquid Emulsions

Acoustic pressure that induced formation of the first visible bubbles was considered the ADV threshold. The data on ADV thresholds for various samples and sonication parameters are presented in Table 2.

TABLE 2
Peak-to-peak pressures (MPa) that correspond to the onset of the ADV
effect in PFP/PEG-PCL and PFP/PEG-PLLA nanoemulsions.

| | | 1% PFP | | 1% PFP | |
|---|---|---|---|---|---|
| | | 0.25% PEG- | | 0.25% PEG- | |
| | | PCL/PBS | | PLLA/PBS | |
| Temperature | Sonication | 1 MHz | 3 MHz | 1 MHz | 3 MHz |
| 22° C. | CW, 12 s | 0.36 | ≦0.36* | 0.57 | ≦0.36 |
| | 20% DC**, 60 s | 0.57 | 0.51 | 0.85 | 0.51 |
| 37° C. | CW, 12 s | 0.3 | ≦0.36 | 0.3 | ≦0.36 |
| | 20% DC, 60 s | 0.44 | ≦0.36 | 0.52 | ≦0.36 |

*the ADV could not be measured because the formation of the bubbles starts immediately after turning on the lowest ultrasound intensity (0.36 MPa) generated by the Omnisound 3000 instrument at 3 MHz.
**DC—Duty cycle.

Bubbles usually form a stream that proceeds from the bottom of a container toward the sample surface. In these experiments, the surface of the samples was located above the sonicated zone. At room temperature, under pulsed ultrasound (1.2 ms pulse with 4.8 ms inter-pulse interval), bubbles were formed during ultrasound-on phase but condensed back into droplets during the inter-pulse interval and therefore oscillated up and down in the ultrasound field. Under CW ultrasound, some bubbles made it to the sample surface and formed foam. It was assumed that after vaporization, the coalescence of some bubbles resulted in a formation of large bubbles, the buoyancy of which allowed them to rise to the sample surface faster than they condensed.

### Effect of the Type of Droplet-Stabilizing Copolymer

For the droplets stabilized by a PEG-PCL copolymer, the ADV threshold was lower than that for the droplets of the same composition stabilized by a PEG-PLLA copolymer. The differences were pronounced for pulsed ultrasound but were small or imperceptible for CW ultrasound. As an example, at room temperature and under 1-MHz ultrasound at a 20% duty cycle (1.2-ms pulse duration and 4.8-ms inter-pulse interval), the ADV threshold for a 1% PFP/0.25% PEG-PCL droplet system was 0.57 MPa compared to 0.85 MPa for the droplets of the same composition stabilized by a PEG-PLLA copolymer (peak-to-peak pressures are presented).

### Effect of Duty Cycle

For both types of the droplets, the ADV threshold depended on the ultrasound duty cycle and was higher for pulsed ultrasound compared to CW ultrasound.

### Effect of Temperature

For both types of the nanodroplet emulsions, the ADV threshold was significantly lower at 37° C. compared to room temperature.

### Effect of Ultrasound Frequency

For both types of bubbles, the ADV threshold was lower for 3-MHz compared to 1-MHz ultrasound.

### ADV in Gel Matrices

Studying the ADV effect in gel matrices is complicated by the unavoidable presence of pre-existing large (hundred micron) bubbles that are formed in the process of sample preparation (FIG. 5A). Analysis of ultrasound images using ImageJ software (a public domain image analysis program developed at the National Institutes of Health) allowed discriminating between pre-existing bubbles and those newly formed by ADV. The number of the latter, if any, was always very low (one or two per sample) even at the highest negative pressure generated by the unfocused transducer (0.61 MPa at 1 MHz, corresponding to 1.18 MPa peak-to-peak pressure). These results do not however rule out transient and reversible formation of primary microbubbles via the ADV in gel matrices. If formed, primary microbubbles were expected to oscillate in ultrasound field thus generating harmonic frequencies, which would confirm their presence. To test this hypothesis, attenuation and cavitation properties of nanodroplets inserted in liquid emulsions and gels (see below) were studied.

In gel matrices sonicated by 1-MHz continuous wave ultrasound at the highest peak-to-peak pressure generated by Omnisound instrument at 1 MHz (1.18 MPa), droplet-to-bubble transition was significantly hampered and occurred predominantly in the immediate vicinity of the pre-existing large bubbles or on their surfaces (FIG. 5B). For comparison, in PBS suspensions, a very intensive formation of bubbles was observed at a pressure as low as 0.57 MPa.

To verify the formation of new bubbles in gel matrices subjected to 1-MHz ultrasound, we measured attenuation of low energy ultrasound in the frequency range from 0.3 to 2.5 MHz. The presence of pre-existing bubbles substantially increased sound attenuation by gel samples (Table 3, fourth column). Some increase of ultrasound attenuation after gel sonication confirmed formation of new bubbles (Table 3, fifth column). Sound attenuation may be caused by sound absorption and/or scattering; the latter increases with increased frequency. A higher sound attenuation at lower frequencies presented in Table 3 suggested a resonance energy absorption by large bubbles occurring at lower frequencies. Resonance energy absorption is expected to facilitate the ADV effect. Therefore a number of experiments were performed with gels sonicated by 90-kHz ultrasound at a peak-to-peak pressure of 0.7 MPa. The effect of a 90-kHz sonication on the nanodroplets inserted in gel matrices was very different from that of 1-MHz ultrasound.

Under 90-kHz ultrasound, the pre-existing bubbles induced a long-range effect on the acoustic droplet vaporization; new bubbles were formed relatively uniformly in the whole volume of the sample (FIG. 5, panel C). These data showed a clear catalytic effect of pre-existing bubbles on the droplet-to-bubble conversion in gel matrices.

TABLE 3
Acoustic parameters of the PFP/PEG-PCL nanodroplets introduced in
the agarose gel.
Phase velocity at 2.5 MHz, m/s

| | Sample | | | |
|---|---|---|---|---|
| | | | PFP/PEG-PCL | PFP/PEG-PCL |
| | | PEG-PCL | nanodroplets | nanodroplet |
| | Pure | micelles | and | sample |
| | Gel, | in the gel, | microbubbles in | sonicated in |
| Sound | 37° C. | 37° C. | the gel, RT | the gel, 37° C. |
| Frequency, | 1509 ± 30 | 1510 ± 30 | 1389 ± 42 | 1404 ± 42 |
| MHz | Attenuation, dB/cm | | | |
| 0.3 | <0.15 | 1.3 | 8.7 | 11.1 |
| 0.5 | <0.15 | 0 | 6.0 | 4.1 |
| 1.7 | <0.15 | 0 | 4.8 | 6.0 |
| 2.5 | <0.15 | 0.5 | 4.0 | 6.3 |

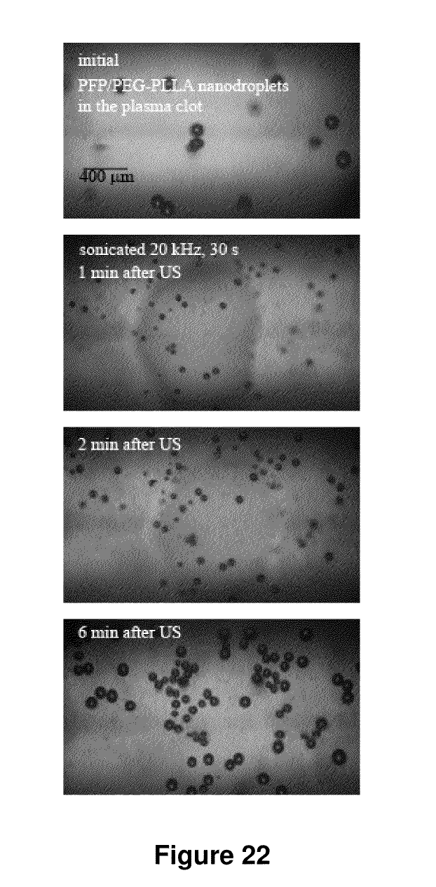

Similar to the data above, FIG. 22 shows (from up to down):

initial gel (i.e. plasma clot) with several PFP/PEG-PLLA microbubbles;
the same gel after sonication with 20-kHz ultrasound; initial microbubbles disappeared, and a much larger number of small microbubbles were formed. These new microbubbles grew with time (next two images), which was probably due to coalescence with surrounding nanodroplets. The growth of newly formed microbubbles is important for enhancing quality of ultrasound contrast that they generate.

### Bubble Cavitation

The interaction of bubbles or droplets with ultrasound is rather complex. Bubble cavitation is believed to be the main mechanism responsible for ultrasound bioeffects. For effective drug delivery, the presence of the nano/microbubbles is extremely beneficial because bubble cavitation triggers release of the encapsulated drug from the carrier and also perturbs cell membranes, thus enhancing intracellular drug uptake. In the present study, cavitation effects were explored for unfocused and focused ultrasound at a frequency of 1 MHz. The appearance and amplitudes of harmonic frequencies and broadband noise was monitored in the fast Fourier transform emission spectra.

The threshold for a subharmonic frequency component was used as a fingerprint of the onset of stable cavitation, whereas the onset of broadband noise characterized inertial cavitation. The latter can generate shock waves and is considered responsible for cell membrane damage and mechanical cell killing by ultrasound. No broadband noise was observed either for liquid emulsions or gels under unfocused ultrasound, suggesting the absence of inertial cavitation at ultrasound pressures generated by Omnisound 3000 instrument (up to the negative pressure of 0.61 MPa). However a second harmonic and subharmonic frequencies were clearly seen in both, liquid emulsions and gels (FIG. 5 A, B), with corresponding threshold pressures being slightly lower in gel samples, presumably due to the presence of pre-existing bubbles that catalyze droplet-to-bubble transition. For the same reason, the pressure dependence of subharmonic amplitude was somewhat smoother for gel samples. The stable cavitation threshold in the gel systems was clearly observed in the focused ultrasound experiments, most probably because sonication was confined to small sample volume with low number or absence of pre-existing bubbles (FIG. 6 B). Cavitation thresholds were close for both studied copolymers.

To characterize inertial cavitation, mean relative amplitudes of broadband noise in the frequency intervals that avoided fundamental, harmonic, and ultraharmonic frequencies were measured.

As mentioned above, no broadband noise was observed in the experiments with unfocused ultrasound while broadband noise was observed in focused ultrasound experiments (FIG. 6 E). For focused ultrasound, the thresholds for generating subharmonic frequencies and broadband noise were close suggesting the onset of inertial cavitation (i.e. unstable growth of microbubbles) as soon as the bubbles started oscillating. In contrast, for 1-MHz unfocused ultrasound at ultrasound pressures employed in other in vivo studies (Rapoport, Gao 2007; Rapoport, Kennedy 2009), only stable cavitation of microbubbles was observed.

Comparing thresholds for ADV and cavitation in liquid emulsions shows that droplet-to-bubble transition via ADV precedes stable and inertial cavitation. The data shown above also indicated that stable microbubble cavitation occurs in both liquid and gel matrices. This suggested that microbubbles are transiently generated and oscillate in gel matrices under the action of unfocused or focused therapeutic ultrasound.

Based on the obtained data, continuous wave 1-MHz ultrasound at a negative pressure of 0.61 MPa corresponding to a peak-to-peak pressure of 1.18 MPa that reliably induced stable cavitation for both droplet-stabilizing copolymers was chosen for in vivo experiments presented in FIGS. 4, 9, and 13-15.

Is Inertial Cavitation a Pre-Requisite for Drug Release from a Carrier?

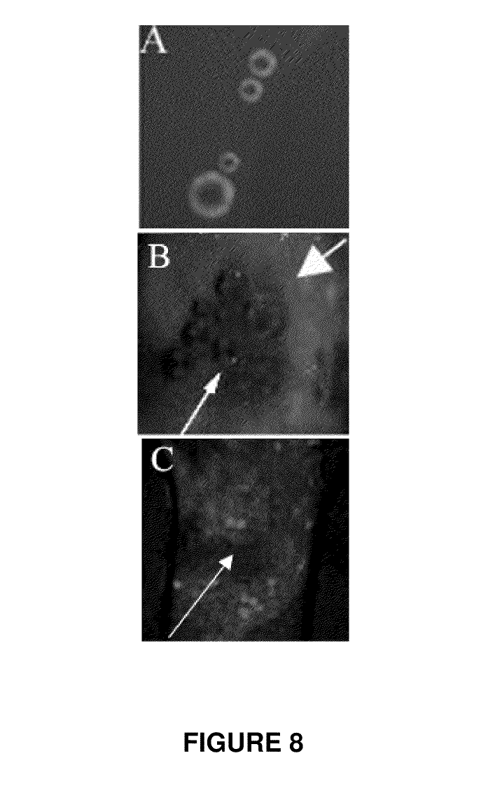

The elucidation of this problem would allow designing optimal ultrasound protocols. Previous data was re-evaluated. These experiments were performed by sonicating a nanodroplet/cell mixture placed in a plastic capillary (internal diameter 340 μm) of a snake mixer slide. FIG. 8A shows that the DOX fluorescence observed in the bubble walls. FIG. 8B shows that fluorescence was substantially reduced after sonication of the bubble mixture with the MDA MB231 breast cancer cell suspension. Note that the bubbles (shown with a thin arrow) did not collapse in the process of sonication, suggesting that they underwent stable cavitation in the ultrasound field (FIG. 8B). After sonication, the cells (shown with a thick arrow) acquired strong fluorescence, indicating that ultrasound induced DOX transfer from the bubble walls into the cells. This process was presumably enhanced by the formation of bubble/cell aggregates triggered by ultrasound; these aggregates are visible in FIG. 8B. FIG. 8C shows another site of the same sample with optical focus on the cells. The data presented above suggested that inertial cavitation was not a strong requirement for drug release from microbubble carriers.

### Ovarian Cancer Chemotherapy by Ultrasound-Activated Drug-Loaded Nanoemulsions

In vivo experiments described below involved ovarian carcinoma bearing mice treated by PTX-loaded nanoemulsions and nanobubbles. The goal of these experiments was verifying nanodroplet/microbubble accumulation in tumor tissue and ultrasound-induced drug release from a carrier. A micellar formulation of PTX, GEN, was chosen because it manifested relatively high effectiveness in treating breast and ovarian cancer. The success of chemotherapy by GEN indicated that the drug was effectively internalized by the tumor cells. It was expected that the transfer of PTX from GEN micelles (29-nm diameter) to nbGEN nanodroplets (about 750-nm diameter) would hamper drug internalization and therefore decrease or eliminate the effect of tumor therapy; the restoration of drug activity under the action of ultrasound would indicate successful drug release from the bubbles. Both assumptions were supported experimentally by the data presented in FIG. 4; after the systemic injection of nbGEN, the unsonicated left tumor grew with the same rate as untreated controlled tumors indicating the lack of therapeutic drug concentration in tumor tissue. The sonicated right tumor effectively regressed. Tumor therapy by empty droplets combined with ultrasound irradiation did not manifest any therapeutic efficacy; the sonicated tumor grew with practically same rate as control tumors (data not shown).

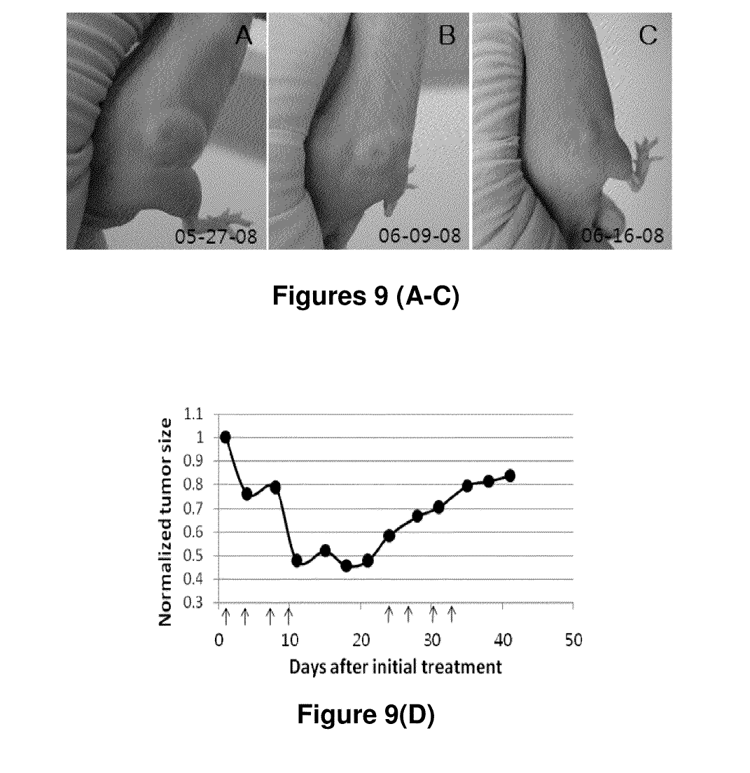

Another example is presented in FIG. 9. For the mouse presented in FIG. 9, the initial tumor volume of 1,650 mm³ dropped about an order of magnitude during the first treatment round. The first photograph (FIG. 9(A)) was taken before the start of the treatment, the second (FIG. 9(B))—two weeks later, i.e. immediately after the last treatment of the first treatment round. The third photograph (FIG. 9(C)) was taken one week after the completion of the first treatment round. However, two weeks after the completion of the first treatment, the residual tumor visible in FIG. 9C manifested signs of re-growth; a second treatment round using the same regimen decreased tumor growth rate to some extent but did not cause the same dramatic tumor regression that occurred during the first treatment round (data not shown). This indicated that residual tumor cells either acquired resistance to PTX or resistant cells were selected during the first treatment round. Normalized tumor growth/regression curve is presented in FIG. 9(D).

### Ultrasound Imaging

Based on the in vitro and in vivo experiments, injection of nanoemulsions into liquid phase, gels, or tumor tissues is accompanied by conversion of some nanodroplets into small bubbles that quickly coalesce to form bubbles of a hundred micron size range (FIG. 10 A, B). This phenomenon can be monitored and quantified using ImageG software due to dramatic differences in droplet and bubble echogenicity. In FIG. 10A, image (a) was recorded immediately after PFP/PEG-PCL nanodroplet injection in Agarose gel; the time difference between image (a) and (b) was 18 s, between image (b) and (c) 25 s, and between image (c) and (d)—30 s. Instrument gain was set at 10 dB. The relatively dark areas in the images shown by thin arrow in image (a) represent droplets (grayscale 104-122); the brightest specks shown with thick arrow in image (a) represent large bubbles formed immediately after nanodroplet injection (grayscale 210-250). With time, the brightness of some dark areas gradually increased from 100 to 160 (shown with arrow in image (d)); these areas presumably represent either droplets growing in size or small bubbles formed by vaporization of droplets within a first minute after injection. The overall brightness was lower and an increase of brightness with time was not observed after the injection of PEG-PLLA stabilized nanodroplets confirming their higher resistance to droplet vaporization.

In the subcutaneous breast tumor tissue injected directly, the grayscale measurements gave values of 85, 133, and 175-250 for the initial tumor tissue before direct nanodroplet injection, for tissue with nanodroplets, and highly echogenic bubbles respectively (FIG. 10 B). The same trend was observed for other tumors; an example for pancreatic cancer injected directly by the nanodroplet formulation of Paclitaxel nbGEN is shown in FIG. 10 C.

After the systemic injections of PEG-PLLA stabilized nanodroplets to ovarian tumors, the grayscale measurements of various regions of interest (ROI) in the tumor tissue gave values of 89, 133, and 154 for the dark, medium bright, and the brightest ROIs respectively, which suggested that the observed tumor-accumulated echogenic particles were droplets and/or small bubbles. Large bubbles with grayscale at or above 200 are rarely observed in ovarian or pancreatic tumors after systemic injections (data not shown).

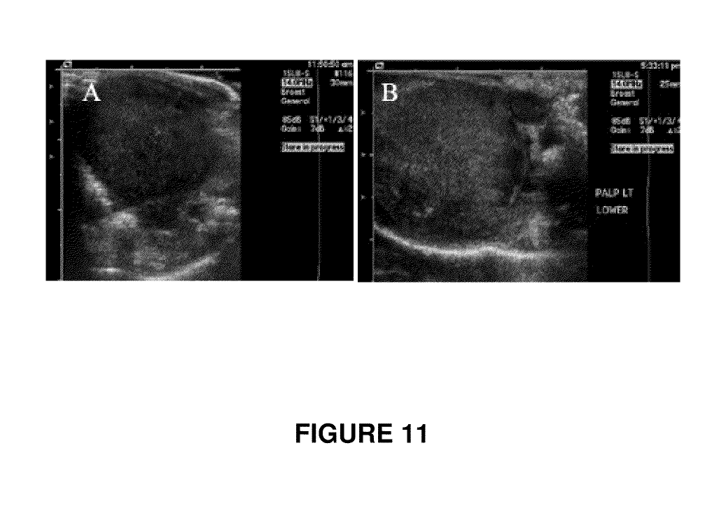

Ultrasound images of the orthotopic (i.e. internal) pancreatic tumor before (left) and 5 h after systemic injection of nbGEN nanodroplets are shown in FIG. 11. The instrument gain was set at 7 dB. The images demonstrate the accumulation of the echogenic particles in the tumor tissue. Mean grayscale values for ROIs of the same area were 44.5±4.0 before the nbGEN injection and 53±5.2 after the injection; the 19% increase was statistically significant (p=0.018, N=9). However, the distribution of the echogenic particles over the particular slice was non-uniform; there was also a significant slice-to-slice variation in the overall echogenicity and distribution of echogenic species.

The extended-time imaging using pancreatic tumors showed the appearance of new bright specks in the tumor tissue two days after systemic injection of the nanoemulsion, which suggested gradual formation of new microbubbles; an increase of the speck echogenicity by about 30% and the formation of new bright specks were also observed after tumor sonication by 90-kHz ultrasound (FIG. 12).

### Ultrasound-Mediated Chemotherapy of Pancreatic Cancer

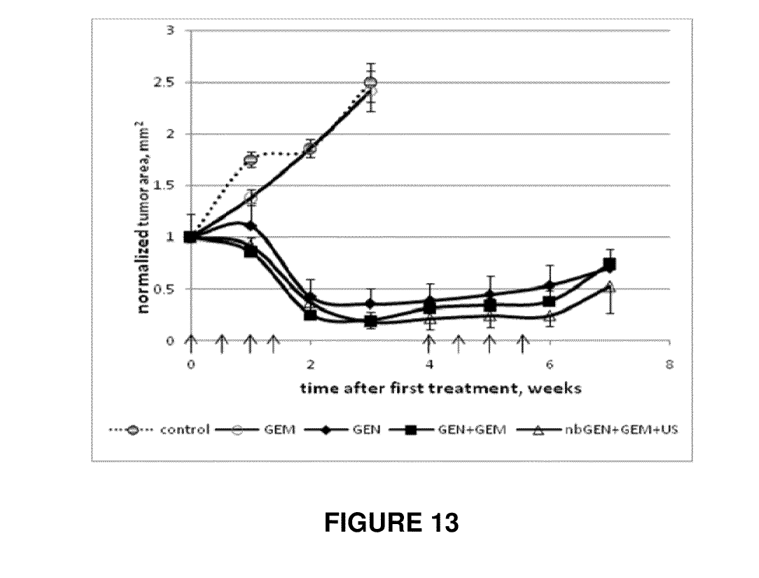

Tumor growth curves for animals treated by Gemcitabine (GEM), micellar formulation of paclitaxel Genexol PM (GEN), combination drug Genexol PM+GEM, and nanoemulsion formulation of paclitaxel nbGEN+GEM and ultrasound are presented in FIG. 13. Good correlation was observed between tumor sizes measured by RFP and ultrasound imaging.

In FIG. 13, animals were treated by Gemcitabine (GEM) (closed circles), micellar encapsulated paclitaxel Genexol PM (GEN) (closed diamonds), combination drug GEM+Genexol PM (closed squares), and combination drug GEM+nanodroplet encapsulated paclitaxel nbGEN combined with continuous wave 1-MHz ultrasound applied for 30 s at 3.4 W/cm² nominal power density to the mouse abdominal area in the pancreas region (open triangles). Mean tumor projection areas plus/minus standard error are presented (N=6). Arrows indicate days of treatment.

As shown in FIG. 13, systemic chemotherapy by nanodroplet encapsulated paclitaxel combined with GEM and ultrasound resulted in dramatic tumor regression. The gemcitabine that was added in this formulation had a very high aqueous solubility, was not internalized by either micelles or droplets and circulated independently.

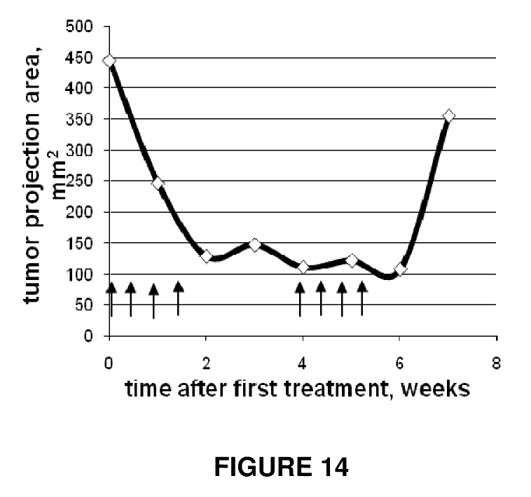

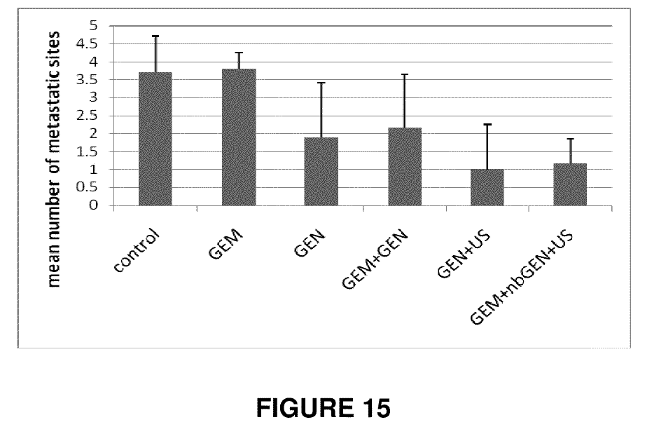

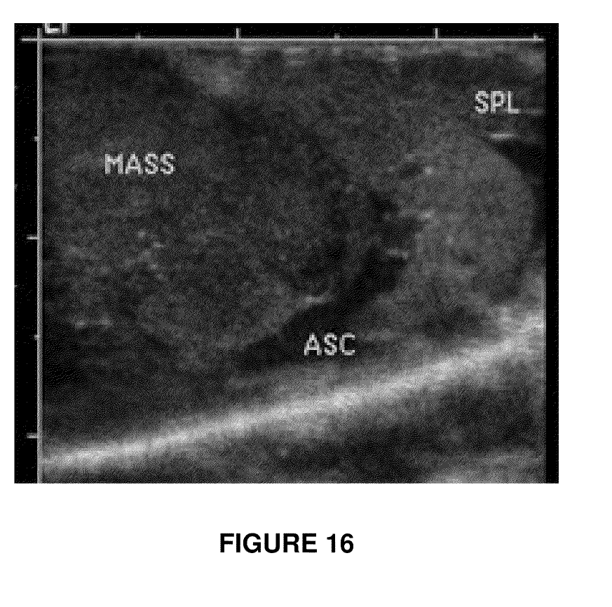

The effect of the combined treatment with nanodroplet encapsulated Genexol PM, GEM and ultrasound was the strongest among the six treatment protocols reported in the experimental section (for instance, P<<0.001 for GEM+nbGEN+ultrasound vs. Genexol PM treatment in a paired T-test) (FIG. 13). The ultrasound effect on the treatment by the nanodroplet encapsulated paclitaxel (nbGEN) was stronger than that on micellar encapsulated paclitaxel (Genexol PM). Even a very large initial tumor effectively regressed under the combined action of nanodroplet encapsulated paclitaxel and ultrasound (FIG. 14). Interestingly, the treatments that involved tumor sonication resulted in a significantly reduced number of metastatic foci and suppression of ascitis formation (FIG. 15). Ascitis was clearly visible in ultrasound images of control or GEM-treated tumors (FIG. 16); no ascitis was found in images of ultrasound-treated tumors or postmortem. This important effect was unexpected.

For all treatment groups, treatment was interrupted for two weeks after the first treatment round. This interruption resulted in tumor re-growth. A second treatment with the same regimen was less effective than the first one. In the Genexol PM and GEM+Genexol PM groups, residual tumors stabilized but did not regress during the second treatment round; only nanodroplet/ultrasound therapy resulted in some regression of re-grown tumors during the second treatment round. This data suggested that either some resistance to paclitaxel developed in the course of the initial treatment or resistant cells were selected during the first treatment round.

With any treatment protocol, local tumor recurrence was observed after completion of treatment. The local recurrence occurred even when the residual tumor could not be resolved by RFP imaging. The possible reasons of this effect are discussed below.

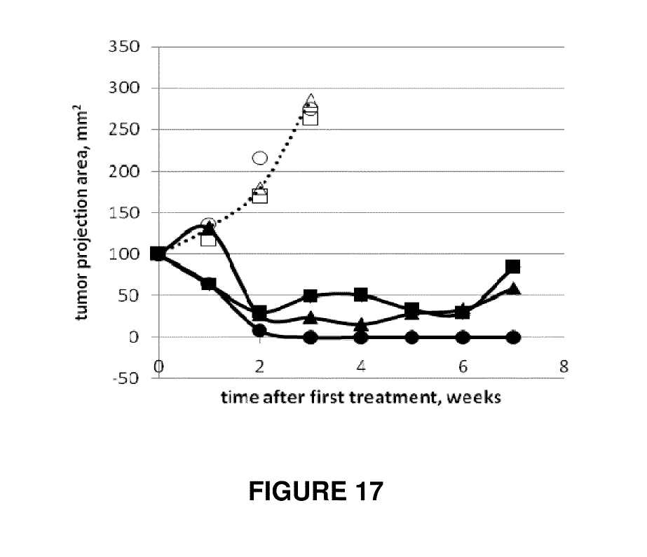

It was noteworthy that the intra-group variations were larger for micellar or nanodroplet encapsulated paclitaxel groups than for control or GEM group. An example for GEM and nbGEN+GEM+US group is shown in FIG. 17.

### Inoperable Breast Cancer Model

Large initial MDA MB231 tumors were grown in nude mice to model inoperable tumors.

Experiment 1: Genexol-loaded nanodroplets were systemically injected into the mice followed by treatments with 90 kHz ultrasound for 4 hours after the injection (without direct injection of empty nanodroplets). Two treatments were administered, but little effect was observed, and the tumor kept growing. Experiment 2: 200 ul Genexol-loaded nanodroplets were systemically injected. Four hours after the systemic administration of the genexol-loaded nanodroplets, 100 ul of empty 1% PFP/0.25% PCL/0.25% L61 nanodroplets were directly injected into the tumor (intratumoral injection). Pluronic L61 was added to the empty nanodroplet composition for several reasons: (1) to increase the size of the empty nanodroplets thus increasing nanodroplet sensitivity to 90-kHz ultrasound and (2) to increase cancerous cell sensitivity to hyperthermia (FIG. 20) and to prevent development of drug resistance. Five minutes after direct injection the tumor was sonicated first by 90 kHz ultrasound (1 min) and then by 3 MHz ultrasound, 20% DC (3 min) As shown in FIG. 21, this treatment was repeated 4 times total (i.e., on day 7, on day 11, on day 15, and on day 18). Very dramatic tumor regression was observed by day 46 (i.e., a little over one month after the last treatment). Experiment 3: Maintaining hyperthermia therapy from day 46 through day 71 (10 treatments were administered during this time period): 100 ul L-61 containing PEG-PLLA micelles (0.25% L-61/0.25% PEG-PLLA micelles (4×25 ul in four tumor locations) were directly injected into the tumor (intratumoral injection), followed by tumor hyperthermia at 43° C. for 5 min (no drug, no sonication). A treatment break was given between days 53 and days 64; however, the tumor kept regressing (FIG. 21 A). Tumor regression stopped after eight treatments suggesting development of heat resistance (most likely due to generation of heat shock proteins). FIG. 21(b) shows an ultrasound image of this tumor; the image was taken a month after direct intratumoral injection of a 100 μl of a 1% PFP/0.25% PEG-PCL nanoemulsion.

### Discussion

The data presented in FIG. 2 indicated that upon mixing a micellar solution of GEN with PFP nanodroplets to form nbGEN, the drug was effectively transferred from the micelles to the droplets, resulting in the decrease of micelle size and increase of droplet size. The size of the micelles (tens of nanometers) and perfluorocarbon nanodroplets (up to 750 nm) favors their localized extravasation through defective tumor microvasculature and accumulation in tumor tissue, which is illustrated schematically in FIG. 1. Particle accumulation in tumor tissue was supported by ultrasound imaging (FIG. 11). The grayscale measurements of ultrasound images suggest that after systemic injection of nanoemulsions, a drug carrier accumulates in the tumor tissue initially in the form of the nanodroplets which convert into microbubbles with time or under the action of therapeutic ultrasound. The nanodroplet vaporization to generate bubbles is highly desirable for both ultrasonography and drug delivery. Bubbles show much higher echogenicity than droplets. Besides producing high ultrasound contrast, bubbles serve as potent enhancers of ultrasound-mediated drug delivery, which droplets do not offer.

As indicated by FIG. 4, without therapeutic ultrasound, the nanodroplets and bubbles strongly retain the loaded drug, which results in a fast growth of the non-sonicated (left) tumor. Strong drug retention in the carrier is important for preventing drug attack on non-targeted tissues. On the other hand, as manifested by a dramatic regression of the sonicated right tumor of FIG. 4, tumor-directed therapeutic ultrasound induces efficient drug release from the nanocarrier, which occurs at least at an order of magnitude lower peak-to-peak pressure compared to that used for tumor ablation; this is important in the context of safety of the developed technology. In vitro experiments (FIG. 8) show that popping of the bubbles is not a strong pre-requisite for the efficient drug transfer from bubbles to cells; stable cavitation of bubbles appears sufficient for drug transfer. However, inertial bubble cavitation may be beneficial because perturbation of cell membranes induced by inertial cavitation increases the intracellular drug uptake. Note that empty droplets combined with ultrasound did not induce any therapeutic effect. The absence of any therapeutic effect of the combined therapy by ultrasound and empty droplets strongly indicates that the therapeutic effect of the drug-loaded droplets is caused by the cytotoxic action of chemotherapeutic drug rather than cancer cell killing by ultrasound. The role of ultrasound consists in effective release of drug from carrier in tumor interstitium and perturbation of cell membranes, which results in enhanced internalization of the released drug. Ultrasound can also increase the inter-endothelial gaps thus enhancing carrier extravasation. These effects were clearly manifested in the results of therapy of pancreatic adenocarcinoma (PDA).

### Gemcitabin Resistance and PTX Sensitivity of Pancreatic MiaPaCa-2 Cells

GEM and paclitaxel have profoundly different mechanisms of action. GEM is the nucleoside analogue and its site of action is in cell nuclei. On the contrary, paclitaxel acts by stabilizing microtubules in cytoplasm thus mechanically preventing cell division. The sensitivity of GEM-resistant MiaPaCa-2 cells to Genexol PM suggested that paclitaxel loaded in PEG-PLLA micelles successfully overcame plasma membrane barriers thus allowing paclitaxel interaction with microtubules. GEM-resistance of MiaPaCa-2 cells may be caused by the action of nuclear pumps that exert no effect on paclitaxel. Other possible mechanisms include several genetic and/or epigenetic alterations; the latter include gene products associated with gemcitabine transport and metabolism.

Low efficacy of GEM has warranted studies of combination drugs. Therefore in this study, GEM was included as a component of a combination formulation with Genexol PM. It was found that combination treatment by GEM+Genexol PM was slightly more effective than Genexol PM alone though the main effect was undoubtedly exerted by Genexol PM (in a paired T-Test, statistically significant differences were manifested between these groups, P=0.01) (FIG. 13). This suggested that Genexol PM may affect intracellular mechanisms involved in GEM inactivation.

### Ultrasound Effects

The most efficient tumor regressions were observed during systemic treatment with nanodroplet encapsulated paclitaxel combined with tumor-directed ultrasound. As suggested by ultrasound imaging, nanodroplets with encapsulated paclitaxel accumulated in tumor tissue. They converted into microbubbles and released their drug load under the action of tumor-directed ultrasound, which resulted in efficient chemotherapy of pancreatic cancer. However, local tumor recurrence was observed during the treatment break or after the completion of treatment and the recurrent tumors proved more resistant to the same treatment regimen indicating developed drug resistance or selection for the resistant cells during the initial treatment. A possible reason for this adverse effect is discussed below.

### Tumor Recurrence

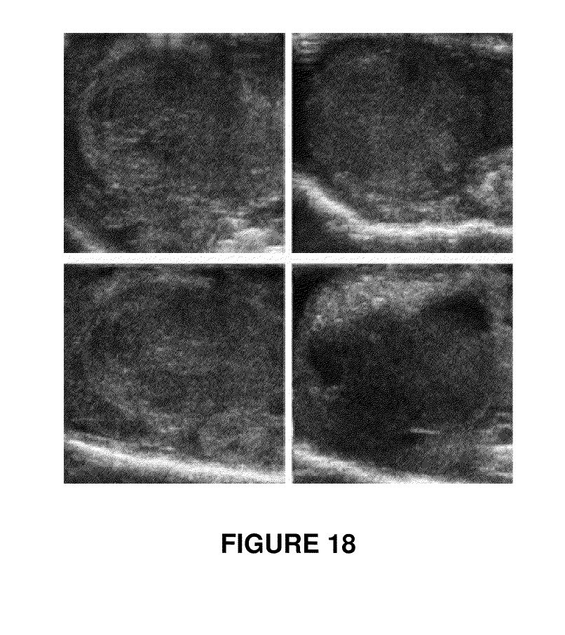

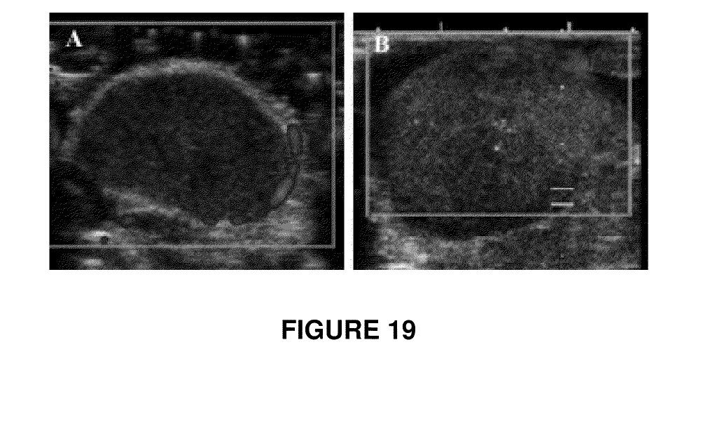

Ultrasound imaging of pancreatic tumors manifested a highly non-uniform distribution of nanodroplets throughout the tumor volume (FIG. 18). This may have been caused by the irregularity in tumor vascularization and distribution of inter-endothelial gaps, which may have resulted in intra-group variability. Doppler images showed the blood vessels that could be resolved by the instrument (hundreds of micron size) localized at the tumor periphery or around the tumor (FIG. 19); smaller capillaries could not be resolved but tumors are known to have irregular vascularization.

After drug release, irregularity of nanoparticle extravasation would result in drug gradients within the tumor volume. If this were the case, some tumor sites may be exposed to sub-therapeutic concentrations of drug, which would favor development of drug resistance. This problem may be solved, at least partly, by tumor sonication. Ultrasound is known to enhance diffusion. Earlier works with micellar doxorubicin have shown that the intratumoral drug distribution was significantly more uniform in sonicated tumors. However, the degree of drug equalization would depend on the mechanical properties of tumor tissue and tumor vascularization. The above problem may be pertinent to any nanoparticle-associated drug delivery modality. To suppress or prevent the development of drug resistance, introduction of MDR-suppressing agents such as Pluronic L-61 into nanoparticle drug formulations may be warranted.

Another intriguing effect that deserves exploration is related to the suppression of pancreatic tumor metastases by ultrasound-mediated chemotherapy with micellar- or nanoemulsion encapsulated paclitaxel (FIG. 15). Recent works have revealed that mechanical forces can profoundly influence cell behavior by affecting cell spread, growth, survival and motility.

For effective PDA therapy, paclitaxel delivery in PFP nanoemulsions may be combined with endoscopic, extracorporeal, or even intraductal ultrasound applicators. The present experiments demonstrated that low-power output application of ultrasound was able to release the drug into tumor tissue.

Double-Frequency Approach to Ultrasound-Mediated Tumor Therapy with Drug-Loaded Nanoemulsions

Measurements of the ADV and cavitation effects showed lower thresholds for PEG-PCL stabilized nanodroplets compared to those stabilized by the same composition of PEG-PLLA. Note that droplet-to-bubble transition is accompanied by increase of particle size. Complete vaporization of the liquid droplet inside the copolymer wall results in a 5-fold increase of a droplet diameter, corresponding to a 25-fold increase of a surface area; this effect depends on elasticity of a bubble wall. The data presented in Table 2 and FIG. 6 suggests that the walls of PEG-PLLA droplets have a higher modulus of elasticity and are stronger than those formed by a PEG-PCL copolymer. Due to this effect, PEG-PCL copolymer requires lower ultrasound energy for droplet-to-bubble transition and bubble cavitation. From this perspective, it appears important to develop techniques that would allow effective droplet-to-bubble transition and bubble cavitation for the nanoemulsions stabilized by strong walls.

The in vitro experiments showed that ultrasound-induced droplet-to-bubble conversion in viscous media is catalyzed by the pre-existing large (hundred microns) microbubbles and is more effective at low ultrasound frequency (90 kHz, 0.7 MPa) compared to 1-MHz or 3-MHz ultrasound (FIG. 5). This may open a new approach to enhancing effectiveness of ultrasound-mediated tumor therapy. Note that large microbubbles of hundred-micron size are usually not observed in tumor tissue after systemic injection of nanoemulsions but can be generated by direct intratumoral injections of nanoemulsions (see FIG. 10). Under the action of low-frequency ultrasound, these microbubbles can catalyze droplet-to-bubble transitions in drug-loaded nanodroplets accumulated in tumor tissue after systemic injection of drug-loaded nanoemulsions, as illustrated in FIG. 5. Micron sized microbubbles that are predominant in the tumor tissue after droplet-to-bubble transition in systemically injected nanoemulsions are not responsive to low-frequency ultrasound but respond to ultrasound in megahertz frequency range.

The new approach to enhancing effectiveness of ultrasound-mediated tumor therapy is discussed below. In the first treatment step, the nanodroplets are injected systemically and given enough time to accumulate in tumor tissue (this is a currently used first treatment step). At the second treatment step, just before application of ultrasound, small volume of nanodroplets (preferably stabilized by PEG-PCL) is injected directly into tumor tissue. At the thirds treatment step, tumor is sonicated by low-frequency ultrasound to induce bubble-catalyzed ADV; some drug may be released from nanodroplets and microbubbles at this sonication stage. Finally at the fourth treatment step, megahertz-frequency ultrasound is applied to tumor in order to effectively release the rest of drug from the small microbubbles formed at the previous stage due to droplet-to-bubble transition in tumor-accumulated nanodroplets. The third and fourth steps can be combined if therapeutic ultrasound of a megahertz frequency range (which allows sharp focusing, in contrast to low-frequency ultrasound) is modulated with a hundred kilohertz frequency component.

It is important to note that microbubbles generated during the direct intratumoral injection of nanodroplets are long lived (see FIG. 21 B), which may allow their multiple re-use for catalyzing droplet-to-bubble transition in systemically injected, tumor accumulated nanodroplets. While systemic injections may be administered weekly, direct intratumoral injection of nanoemulsions may be performed once at the beginning of therapy and then repeated in a month or at the start of the next treatment round.

Combining drug delivery in the nanoemulsions that are accumulated in tumors and converted into microbubbles in situ with ultrasound-triggered drug release may present efficient double-targeting chemotherapeutic modality for solid tumors.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A method for treating a tumor comprising the steps:
(a) contacting the tumor with a therapeutic agent encapsulated in a first nanoemulsion; and
(b) exposing the tumor to a first ultrasonic radiation in an amount less than 100 kHz.

2. The method of claim 1, further comprising injecting a second nanoemulsion into the tumor by intratumoral injection after step (a) but before step (b).

3. The method of claim 1, further comprising exposing the tumor after the first ultrasonic radiation step to a second ultrasonic radiation from about 1 MHz to about 5 MHz.

4. The method of claim 3, wherein the second ultrasonic radiation comprises from about 1 MHz to about 3 MHz.

5. The method of claim 1, wherein the first ultrasonic radiation is from about 20 kHz to about 90 kHz.

6. The method of claim 1, wherein the first ultrasonic radiation is about 90 kHz.

7. The method of claim 1, wherein the therapeutic agent comprises a chemotherapeutic drug.

8. The method of claim 1, wherein the therapeutic agent comprises paclitaxel, doxorubicin, gemcitabine, adriamycin, cisplatin, taxol, methotrexate, 5-fluorouracil, betulinic acid, amphotericin B, diazepam, nystatin, propofol, testosterone, estrogen, prednisolone, prednisone, 2,3 mercaptopropanol, progesterone, a MDR suppressing agent, or any combination thereof.

9. The method of claim 1, wherein the therapeutic agent comprises paclitaxel, doxorubicin, gemcitabine, or any combination thereof.

10. The method of claim 1, wherein the tumor is a multidrug resistant tumor.

11. The method of claim 1, wherein the tumor is breast cancer, pancreatic cancer, ovarian cancer, or a combination thereof.

12. The method of claim 11, wherein the pancreatic cancer is pancreatic ductal cancer.

13. The method of claim 1, wherein the nanoemulsion has a diameter from about 20 nm to less than 1000 nm.

14. The method of claim 1, wherein the nanoemulsion comprises a nanosized micelle, a nanodroplet, or a combination thereof.

15. The method of claim 14, wherein the micelle has a diameter from about 20 nm to about 100 nm in diameter.

16. The method of claim 14, wherein the nanodroplet has a diameter less than or equal to 1000 nm.

17. The method of claim 1, wherein the nanoemulsion comprises a block copolymer, a halogen containing compound, a Pluronic, or a combination thereof.

18. The method of claim 17, wherein the block copolymer comprises a poly(alkylene oxide) and a second polymer.

19. The method of claim 18, wherein the poly(alkylene oxide) comprises a polyethylene oxide, a polypropylene oxide, a polybutylene oxide, a polypentylene oxide, or a combination thereof.

20. The method of claim 18, wherein the poly(alkylene oxide) is polyethylene oxide.

21. The method of claim 18, wherein the second polymer comprises a hydrophobic polymer.

22. The method of claim 18, wherein the second polymer comprises a polymer of lactic acid, a polylactone, or a combination thereof.

23. The method of claim 18, wherein the second polymer comprises poly(l)lactic acid, poly(d)lactic acid, or a combination thereof.

24. The method of claim 18, wherein the second polymer is polycaprolactone.

25. The method of claim 17, wherein the halogen containing compound comprises a fluoro containing compound.

26. The method of claim 17, wherein the halogen containing compound comprises a perfluorocarbon.

27. The method of claim 17, wherein the halogen containing compound is perfluoropentane.

28. The method of claim 1, wherein the nanoemulsion comprises a polyethylene glycol poly(l)lactic acid block copolymer, a perfluoropentane, a therapeutic agent, or any combination thereof.

29. The method of claim 1, wherein the nanoemulsion comprises a polyethylene glycol polycaprolactone block copolymer, a perfluoropentane, a therapeutic agent or any combination thereof.

30. The method of claim 1, wherein the nanoemulsion comprises a polyethylene glycol poly(l)lactic acid block copolymer, a perfluoropentane, and a therapeutic agent, wherein the therapeutic agent comprises paclitaxel, doxorubicin, gemcitabine, or any combination thereof.

31. The method of claim 1, wherein the nanoemulsion comprises a polyethylene glycol polycaprolactone block copolymer, a perfluoropentane, and a therapeutic agent, wherein the therapeutic agent comprises paclitaxel, doxorubicin, gemcitabine, or any combination thereof.

32. The method of claim 1, wherein hyperthermia is administered to the tumor concurrently with or after step (c).

33. The method of claim 3, wherein hyperthermia is administered to the tumor concurrently with or after exposing the tumor to the second ultrasonic radiation.

34. A method of treating a cancer in a subject comprising the steps:
(a) injecting a therapeutic agent encapsulated in a first nanoemulsion into the subject;
(b) administering a first ultrasonic radiation of less than 300 kHz to the tumor; and
(c) administering a second ultrasonic radiation from about 1 MHz to about 5 MHz to the tumor.

35. The method of claim 34, further comprising injecting a second nanoemulsion into the tumor by intratumoral injection after step (a) but before step (b).

36. method of claim 34, wherein the second ultrasonic radiation is from about 1 MHz to about 3 MHz.

37. The method of claim 34, wherein the first ultrasonic radiation is less than 100 kHz.

38. The method of claim 34, wherein hyperthermia is administered to the tumor concurrently with or after step (c).
